(19) United States
(12) Patent Application Publication
(10) Pub. No.: US 2010/0011656 A1
(43) Pub. Date: Jan. 21, 2010

(54) USE OF RYLENES AS MARKERS FOR LIQUIDS

(75) Inventors: Thomas Gessner, Heidelberg (DE); Sophia Ebert, Mannheim (DE); Ruediger Sens, Ludwigshafen (DE); Martin Koenemann, Mannheim (DE); Wolfgang Ahlers, Lambsheim (DE); Christos Vamvakaris, Mannheim (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/280,577

(22) PCT Filed: Feb. 23, 2007
(86) PCT No.: PCT/EP2007/051745

§ 371 Date: Aug. 25, 2008

(30) Foreign Application Priority Data
Mar. 1, 2006 (EP) 06110552.4

(51) Int. Cl. C10L 1/232; C07D 471/06; G01N 21/76

(52) U.S. Cl. 44/338; 546/66; 546/37; 436/172

(57) ABSTRACT

The invention relates to the use of rylene derivatives of the general formula (I) as markers for liquids,

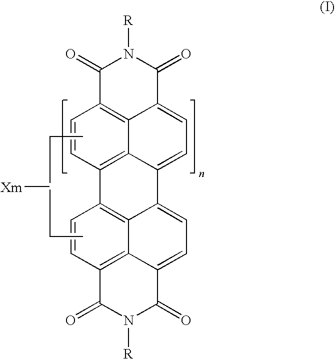

where the symbols and indices have the meanings given in the specification.

The invention relates to the use of rylene compounds as markers for liquids, especially of oils such as mineral oils, liquids comprising such rylene compounds, and to a process for marking liquids and for detecting marked liquids.

It is frequently necessary to mark liquids in order to be able to identify the liquids thus marked subsequently by means of suitable detection methods. For example, liquid product streams can be marked in industrial plants such as mineral oil refineries and thus be tracked.

The marking of mineral oils, for example the different qualities of diesel and gasoline fuels, is of great economic significance. In this way, heating oil, which usually enjoys tax privileges, can be differentiated from the generally more highly taxed diesel oil.

Since the marking should normally be invisible to the human eye, markers are correspondingly used which either absorb and/or emit radiation outside the visible region of the spectrum or which can be detected even in a small amount without changing the visual appearance of the marked compared to the unmarked liquid owing to their intrinsic color in the visible region of the spectrum.

When the markers are to be detected directly in the marked mineral oil sample by means of a spectroscopic method, it is advantageously possible to use markers whose absorption and/or fluorescence is within the wavelength range above about 600 nm, since mineral oils generally themselves have high absorption and/or fluorescence below this wavelength owing to their aromatics content.

For example, WO 94/02570 describes a process for marking hydrocarbons or mineral oils with markers which absorb or fluoresce in the infrared region of the spectrum and can thus be detected.

The marking of mineral oils with markers which are not visible to the human eye and their spectroscopic detection is detailed in the document EP 1 001 003.

Acid-extractable markers for hydrocarbons or mineral oils are described, for example, in EP-A 0 519 270 and base-extractable markers, for example, in WO 96/32462, the detection of the markers generally being effected visually by virtue of their concentration in the extract and/or by virtue of their reaction with suitable reagents to form colored reaction products.

Although good results are already achieved with the known markers, there is still a broad need for improvements, for example concerning the reduction in the amount of markers, their stability toward further additives or their possible harmful influences, for example on the fuel intake and exhaust gas outlet region of internal combustion engines.

It is thus an object of the invention to provide further compounds for use as markers for liquids, especially for mineral oils, which avoid the known disadvantages of the prior art at least in some regions and have favorable application properties such as good solubility in the liquids and good detectability even in ultrasmall amounts in the correspondingly marked liquids.

It has been found that certain rylene compounds are outstandingly suitable as markers for liquids, especially oils, such as mineral oils.

The invention therefore provides for the use of rylene derivatives of the general formula (I) as markers for liquids,

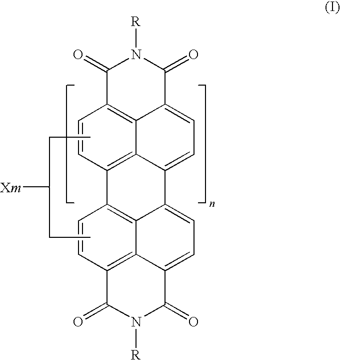

where the symbols and indices are defined as follows

n is an integer from 0 to 3;

m is an integer from 0 to 2 when n=0,

m is an integer from 0 to 4 when n=1,

m is an integer from 0 to 4 when n=2,

m is an integer from 0 to 6 when n=3;

X is the same or different and is
halogen
or
C₁-C₂₀-alkyl, C₃-C₂₀-alken-2-yl or C₃-C₂₀-alkyn-2-yl, whose alkyl chain may in each case be interrupted by one or more —O—, —S—, —NR¹—, —CO— and/or —SO₂— moieties and which may be mono- or polysubstituted by cyano, C₁-C₆-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic;
or
aryloxy, arylthio, hetaryloxy or hetarylthio, to each of which may be fused further saturated or unsaturated 5- to 7-membered rings whose carbon skeleton may be interrupted by one or more —O—, —S—, —NR¹—, —N═CR¹—, —CO—, —SO— and/or —SO₂— moieties, where the entire ring system may be mono- or polysubstituted by the alkyl radicals (1), cycloalkyl radicals (2), aryl or hetaryl radicals (3) mentioned for R and/or the (i) and/or (iv) radicals mentioned there;
or
a 5- to 9-membered ring which is bonded via a nitrogen atom and whose carbon chain may be interrupted by one or more —O—, —S—, —NR¹—, —CO— and/or —SO₂— moieties, to which may be fused one or two unsaturated or saturated 4- to 8-membered rings whose carbon chain may likewise be interrupted by these moieties and/or —N═, where the entire ring system may be mono- or polysubstituted by: hydroxyl, nitro, —NHR², carboxyl, —COOR², —CONR²R³ or —NR²COR³;
C₁-C₃₀-alkyl whose carbon chain may be interrupted by one or more —O—, —S—, —NR¹—, —CO— and/or —SO₂— moieties and which may be mono- or polysubstituted by cyano, hydroxyl, nitro, C₁-C₆-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic;
C₅-C₈-cycloalkyl whose carbon skeleton may be interrupted by one or more —O—, —S— and/or —NR¹— moieties and/or which may be mono- or polysubstituted by C₁-C₆-alkyl;
aryl or hetaryl, each of which may be mono- or polysubstituted by C₁-C₁₈-alkyl, C₁-C₆-alkoxy, cyano, nitro, halogen, —CONR²R³, —NR²COR³, —SO₂NR²R³ and/or aryl- or hetarylazo, each of which may be substituted by C₁-C₁₀-alkyl, C₁-C₆-alkoxy or cyano,
or
C₁-C₁₂-alkoxy, C₁-C₁₂-alkylthio or NR³R⁴;

R¹ is the same or different and is hydrogen or C₁-C₁₈-alkyl,

R², R³, R⁴ are the same or different and are each hydrogen,
C₁-C₁₈-alkyl whose carbon chain may be interrupted by one or more —O—, —S—, —CO—, —SO— and/or —SO₂— moieties and which may be mono- or polysubstituted by C₁-C₁₂-alkoxy, C₁-C₆-alkylthio, hydroxyl, mercapto, halogen, cyano, nitro and/or —COOR¹;
aryl or hetaryl, to each of which may be fused further saturated or unsaturated 5- to 7-membered rings whose carbon skeleton may be interrupted by one or more —O—, —S—, —CO— and/or —SO₂— moieties, where the entire ring system may be mono- or polysubstituted by C₁-C₁₂-alkyl and/or the aforementioned radicals mentioned as substituents for alkyl;
and

R is the same or different and is hydrogen,
(1) C₁-C₃₀-alkyl whose carbon chain may be interrupted by one or more —O—, —S—, —NR¹—, —N═CR¹—, —C≡C—, —CR¹═CR¹—, —CO—, —SO— and/or —SO₂— moieties and which may be mono- or polysubstituted by:
(i) C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR² and/or —SO₃R²;
(ii) aryl or hetaryl, to which may be fused further saturated or unsaturated 5- to 7-membered rings whose carbon skeleton may be interrupted by one or more —O—, —S—, —NR¹—, —N═CR¹—, —CR¹═CR¹—, —CO—, —SO— and/or —SO₂— moieties, where the entire ring system may be mono- or polysubstituted by: C₁-C₁₈-alkyl, C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR², —SO₃R², aryl and/or hetaryl, each of which may be substituted by C₁-C₁₈-alkyl, C₁-C₁₂-alkoxy, hydroxyl, mercapto, halogen, cyano, nitro, —NR²R³, —NR²COR³, —CONR²R³, —SO₂NR²R³, —COOR² and/or —SO₃R²;
(iii) C₃-C₈-cycloalkyl, whose carbon skeleton may be interrupted by one or more —O—, —S—, —NR¹—, —N═CR¹—, —CR¹═CR¹—, —CO—, —SO— and/or —SO₂— moieties and to which may be fused further saturated or unsaturated 5- to 7-membered rings whose carbon skeleton may be interrupted by one or more —O—, —S—, —NR¹—, —N═CR¹—, —CR¹═CR¹—, —CO—, —SO— and/or —SO₂— moieties, where the entire ring system may be mono- or polysubstituted by: C₁-C₁₈-alkyl, C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR² and/or —SO₃R²;
(iv) a —U-aryl radical which may be mono- or polysubstituted by the above radicals mentioned as substituents for the aryl radicals (ii), where U is an —O—, —S—, —NR¹—, —CO—, —SO— or —SO₂— moiety;
(2) C₃-C₈-cycloalkyl to which may be fused further saturated or unsaturated 5- to 7-membered rings whose carbon skeleton may be interrupted by one or more —O—, —S—, —NR¹—, —N═CR¹—, —CR¹═CR¹—, —CO—, —SO— and/or —SO₂— moieties, where the entire ring system may be mono- or polysubstituted by: the (i), (ii), (iii), (iv) radicals and/or
(v) C₁-C₃₀-alkyl, whose carbon chain may be interrupted by one or more —O—, —S—, —NR¹—, —N═CR¹—, —C≡C—, —CR¹═CR¹—, —CO—, —SO— and/or —SO₂— moieties and which may be mono- or polysubstituted by: C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR², —SO₃R², aryl and/or saturated or unsaturated C₄-C₇-cycloalkyl whose carbon skeleton may be interrupted by one or more —O—, —S—, —NR¹—, —N═CR¹—, —CR¹═CR¹—, —CO—, —SO— and/or —SO₂— moieties, where the aryl and cycloalkyl radicals may each be mono- or polysubstituted by C₁-C₁₈-alkyl and/or the above radicals mentioned as substituents for alkyl;
(3) aryl or hetaryl, to which may be fused further saturated or unsaturated 5- to 7-membered rings whose carbon skeleton may be interrupted by one or more —O—, —S—, —NR¹—, —N═CR¹—, —C≡C—, —CR¹═CR¹—, —CO—, —SO— and/or —SO₂— moieties, where the entire ring system may be substituted by the (i), (ii), (iii), (iv), (v) radicals and/or aryl- and/or hetarylazo, each of which may be substituted by C₁-C₁₀-alkyl, C₁-C₆-alkoxy and/or cyano.

In the context of the invention, the term rylenes includes naphthalenes (n=0).

All alkyl groups occurring in the formula (I) may be either straight-chain or branched. Aromatic radicals which are substituted generally have up to 3, preferably 1 or 2, of the substituents mentioned.

Examples of suitable X, R, R¹ to R⁴ radicals and, if appropriate, of their particular substituents include:
methyl, ethyl, propyl, isopropyl, butyl, isobutyl, tert-butyl, pentyl, isopentyl, neopentyl, tert-pentyl, hexyl, 2-methylpentyl, heptyl, 1-ethylpentyl, octyl, 2-ethylhexyl, isooctyl, nonyl, isononyl, decyl, isodecyl, undecyl, dodecyl, tridecyl, isotridecyl, tetradecyl, pentadecyl, hexadecyl, heptadecyl, octadecyl, nonadecyl and eicosyl (the above terms isooctyl, isononyl, isodecyl and isotridecyl are trivial terms and stem from the alcohols obtained by the oxo process);
2-methoxyethyl, 2-ethoxyethyl, 2-propoxyethyl, 2-isopropoxyethyl, 2-butoxyethyl, 2- and 3-methoxypropyl, 2- and 3-ethoxypropyl, 2- and 3-propoxypropyl, 2- and 3-butoxypropyl, 2- and 4-methoxybutyl, 2- and 4-ethoxybutyl, 2- and 4-propoxybutyl, 3,6-dioxaheptyl, 3,6-dioxaoctyl, 4,8-dioxanonyl, 3,7-dioxaoctyl, 3,7-dioxanonyl, 4,7-dioxaoctyl, -4,7-dioxanonyl, 2- and 4-butoxybutyl, 4,8-dioxadecyl, 3,6,9-trioxadecyl, 3,6,9-trioxaundecyl, 3,6,9-trioxadodecyl, 3,6,9,12-tetraoxatridecyl and 3,6,9,12-tetraoxatetradecyl;
2-methylthioethyl, 2-ethylthioethyl, 2-propylthioethyl, 2-isopropylthioethyl, 2-butylthioethyl, 2- and 3-methylthiopropyl, 2- and 3-ethylthiopropyl, 2- and 3-propylthiopropyl, 2- and 3-butylthiopropyl, 2- and 4-methylthiobutyl, 2- and 4-ethylthiobutyl, 2- and 4-propylthiobutyl, 3,6-dithiaheptyl, 3,6-dithiaoctyl, 4,8-dithianonyl, 3,7-dithiaoctyl, 3,7-dithianonyl, 2- and 4-butylthiobutyl, 4,8-dithiadecyl, 3,6,9-trithiadecyl, 3,6,9-trithiaundecyl, 3,6,9-trithiadodecyl, 3,6,9,12-tetrathiatridecyl and 3,6,9,12-tetrathiatetradecyl;
2-monomethyl- and 2-monoethylaminoethyl, 2-dimethylaminoethyl, 2- and 3-dimethylaminopropyl, 3-monoisopropylaminopropyl, 2- and 4-monopropylaminobutyl, 2- and 4-dimethylaminobutyl, 6-methyl-3,6-diazaheptyl, 3,6-dimethyl-3,6-diazaheptyl, 3,6-diazaoctyl, 3,6-dimethyl-3,6-diazaoctyl, 9-methyl-3,6,9-triazadecyl, 3,6,9-trimethyl-3,6,9-triazadecyl, 3,6,9-triazaundecyl, 3,6,9-trimethyl-3,6,9-triazaundecyl, 12-methyl-3,6,9,12-tetraazatridecyl and 3,6,9,12-tetramethyl-3,6,9,12-tetraazatridecyl;
(1-ethylethylidene)aminoethylene, (1-ethylethylidene)aminopropylene, (1-ethyl-ethylidene)aminobutylene, (1-ethylethylidene)aminodecylene and (1-ethylethylidene)-aminododecylene;
propan-2-on-1-yl, butan-3-on-1-yl, butan-3-on-2-yl and 2-ethylpentan-3-on-1-yl;
2-methylsulfoxidoethyl, 2-ethylsulfoxidoethyl, 2-propylsulfoxidoethyl, 2-isopropylsulfoxidoethyl, 2-butylsulfoxidoethyl, 2- and 3-methylsulfoxidopropyl, 2- and 3-ethylsulfoxidopropyl, 2- and 3-propylsulfoxidopropyl, 2- and 3-butylsulfoxidopropyl, 2- and 4-methylsulfoxidobutyl, 2- and 4-ethylsulfoxidobutyl, 2- and 4-propylsulfoxidobutyl and 4-butylsulfoxidobutyl;
2-methylsulfonylethyl, 2-ethylsulfonylethyl, 2-propylsulfonylethyl, 2-isopropylsulfonylethyl, 2-butylsulfonylethyl, 2- and 3-methylsulfonylpropyl, 2- and 3-ethylsulfonylpropyl, 2- and 3-propylsulfonylpropyl, 2- and 3-butylsulfonylpropyl, 2- and 4-methylsulfonylbutyl, 2- and 4-ethylsulfonylbutyl, 2- and 4-propylsulfonylbutyl and 4-butylsulfonylbutyl;
carboxymethyl, 2-carboxyethyl, 3-carboxypropyl, 4-carboxybutyl, 5-carboxypentyl, 6-carboxyhexyl, 8-carboxyoctyl, 10-carboxydecyl, 12-carboxydodecyl and 14-carboxytetradecyl;
sulfomethyl, 2-sulfoethyl, 3-sulfopropyl, 4-sulfobutyl, 5-sulfopentyl, 6-sulfohexyl, 8-sulfooctyl, 10-sulfodecyl, 12-sulfododecyl and 14-sulfotetradecyl;
2-hydroxyethyl, 2- and 3-hydroxypropyl, 1-hydroxyprop-2-yl, 3- and 4-hydroxybutyl, 1-hydroxybut-2-yl and 8-hydroxy-4-oxaoctyl;
2-cyanoethyl, 3-cyanopropyl, 3- and 4-cyanobutyl, 2-methyl-3-ethyl-3-cyanopropyl, 7-cyano-7-ethylheptyl and 4,7-dimethyl-7-cyanoheptyl;
2-chloroethyl, 2- and 3-chloropropyl, 2-, 3- and 4-chlorobutyl, 2-bromoethyl, 2- and 3-bromopropyl and 2-, 3- and 4-bromobutyl;
2-nitroethyl, 2- and 3-nitropropyl and 2-, 3- and 4-nitrobutyl;
methoxy, ethoxy, propoxy, isopropoxy, butoxy, isobutoxy, sec-butoxy, tert-butoxy, pentoxy, isopentoxy, neopentoxy, tert-pentoxy and hexoxy;
methylthio, ethylthio, propylthio, isopropylthio, butylthio, isobutylthio, sec-butylthio, tert-butylthio, pentylthio, isopentylthio, neopentylthio, tert-pentylthio and hexylthio;
methylamino, ethylamino, propylamino, isopropylamino, butylamino, isobutylamino, pentylamino, hexylamino, dimethylamino, methylethylamino, diethylamino, dipropylamino, diisopropylamino, dibutylamino, diisobutylamino, dipentylamino, dihexylamino, dicyclopentylamino, dicyclohexylamino, dicycloheptylamino, diphenylamino and dibenzylamino;
formylamino, acetylamino, propionylamino and benzoylamino;
carbamoyl, methylaminocarbonyl, ethylaminocarbonyl, propylaminocarbonyl, butylaminocarbonyl, pentylaminocarbonyl, hexylaminocarbonyl, heptylaminocarbonyl, octylaminocarbonyl, nonylaminocarbonyl, decylaminocarbonyl and phenylaminocarbonyl;
aminosulfonyl, N,N-dimethylaminosulfonyl, N,N-diethylaminosulfonyl, N,N-dipropylaminosulfonyl, N,N-diisopropylaminosulfonyl, N,N-dibutylaminosulfonyl, N,N-diisobutylaminosulfonyl, N,N-di-sec-butylaminosulfonyl, N,N-di-tert-butylaminosulfonyl, N,N-dipentylaminosulfonyl, N,N-dihexylaminosulfonyl, N,N-diheptylaminosulfonyl, N,N-dioctylaminosulfonyl, N,N-dinonylaminosulfonyl, N,N-didecylaminosulfonyl, N,N-didodecylaminosulfonyl, N-methyl-N-ethylaminosulfonyl, N-methyl-N-dodecylaminosulfonyl, N-dodecylaminosulfonyl, (N,N-dimethylamino)ethylaminosulfonyl, N,N-(propoxyethyl)dodecylaminosulfonyl, N,N-diphenylaminosulfonyl, N,N-(4-tert-butylphenyl)octadecylaminosulfonyl and N,N-bis(4-chlorophenyl)aminosulfonyl;
methoxycarbonyl, ethoxycarbonyl, propoxycarbonyl, isopropoxycarbonyl, hexoxycarbonyl, dodecyloxycarbonyl, octadecyloxycarbonyl, phenoxycarbonyl, (4-tert-butylphenoxy)carbonyl and (4-chlorophenoxy)carbonyl;
methoxysulfonyl, ethoxysulfonyl, propoxysulfonyl, isopropoxysulfonyl, butoxysulfonyl, isobutoxysulfonyl, tert-butoxysulfonyl, hexoxysulfonyl, dodecyloxysulfonyl, octadecyloxysulfonyl, phenoxysulfonyl, 1- and 2-naphthyloxysulfonyl, (4-tert-butylphenoxy)-sulfonyl and (4-chlorophenoxy)sulfonyl;
chlorine, bromine and iodine;
phenylazo, 2-naphthylazo, 2-pyridylazo and 2-pyrimidylazo;
cyclopropyl, cyclobutyl, cyclopentyl, 2- and 3-methylcyclopentyl, 2- and 3-ethylcyclopentyl, cyclohexyl, 2-, 3- and 4-methylcyclohexyl, 2-, 3- and 4-ethylcyclohexyl, 3- and 4-propylcyclohexyl, 3- and 4-isopropylcyclohexyl, 3- and 4-butylcyclohexyl, 3- and 4-sec-butylcyclohexyl, 3- and 4-tert-butylcyclohexyl, cycloheptyl, 2-, 3- and 4-methylcycloheptyl, 2-, 3- and 4-ethylcycloheptyl, 3- and 4-propylcycloheptyl, 3- and 4-isopropylcycloheptyl, 3- and 4-butylcycloheptyl, 3- and 4-sec-butylcycloheptyl, 3- and 4-tert-butylcycloheptyl, cyclooctyl, 2-, 3-, 4- and 5-methylcyclooctyl, 2-, 3-, 4- and 5-ethylcyclooctyl and 3-, 4- and 5-propylcyclooctyl; 3- and 4-hydroxycyclohexyl, 3- and 4-nitrocyclohexyl and 3- and 4-chlorocyclohexyl;
1-, 2- and 3-cyclopentenyl, 1-, 2-, 3- and 4-cyclohexenyl, 1-, 2- and 3-cycloheptenyl and 1-, 2-, 3- and 4-cyclooctenyl;
2-dioxanyl, 1-morpholinyl, 1-thiomorpholinyl, 2- and 3-tetrahydrofuryl, 1-, 2- and 3-pyrrolidinyl, 1-piperazyl, 1-diketopiperazyl and 1-, 2-, 3- and 4-piperidyl;
phenyl, 2-naphthyl, 2- and 3-pyrryl, 2-, 3- and 4-pyridyl, 2-, 4- and 5-pyrimidyl, 3-, 4- and 5-pyrazolyl, 2-, 4- and 5-imidazolyl, 2-, 4- and 5-thiazolyl, 3-(1,2,4-triazyl), 2-(1,3,5-triazyl), 6-quinaldyl, 3-, 5-, 6- and 8-quinolinyl, 2-benzoxazolyl, 2-benzothiazolyl, 5-benzothiadiazolyl, 2- and 5-benzimidazolyl and 1- and 5-isoquinolyl;
1-, 2-, 3-, 4-, 5-, 6- and 7-indolyl, 1-, 2-, 3-, 4-, 5-, 6- and 7-isoindolyl, 5-(4-methylisoindolyl), 5-(4-phenylisoindolyl), 1-, 2-, 4-, 6-, 7- and 8-(1,2,3,4-tetrahydroisoquinolin 3-(5-phenyl)-(1,2,3,4-tetrahydroisoquinolinyl), 5-(3-dodecyl-(1,2,3,4-tetrahydroisoquinolinyl), 1-, 2-, 3-, 4-, 5-, 6-, 7- and 8-(1,2,3,4-tetrahydroquinolinyl) and 2-, 3-, 4-, 5-, 6-, 7- and 8-chromanyl, 2-, 4- and 7-quinolinyl, 2-(4-phenylquinolinyl) and 2-(5-ethylquinolinyl);
2-, 3- and 4-methylphenyl, 2,4-, 3,5- and 2,6-dimethylphenyl, 2,4,6-trimethylphenyl, 2-, 3- and 4-ethylphenyl, 2,4-, 3,5- and 2,6-diethylphenyl, 2,4,6-triethylphenyl, 2-, 3- and 4-propylphenyl, 2,4-, 3,5- and 2,6-dipropylphenyl, 2,4,6-tripropylphenyl, 2-, 3- and 4-isopropylphenyl, 2,4-, 3,5- and 2,6-diisopropylphenyl, 2,4,6-triisopropylphenyl, 2-, 3- and 4-butylphenyl, 2,4-, 3,5- and 2,6-dibutylphenyl, 2,4,6-tributylphenyl, 2-, 3- and 4-isobutylphenyl, 2,4-, 3,5- and 2,6-diisobutylphenyl, 2,4,6-triisobutylphenyl, 2-, 3- and 4-sec-butylphenyl, 2,4-, 3,5- and 2,6-di-sec-butylphenyl and 2,4,6-tri-sec-butylphenyl; 2-, 3- and 4-methoxyphenyl, 2,4-, 3,5- and 2,6-dimethoxyphenyl, 2,4,6-trimethoxyphenyl, 2-, 3- and 4-ethoxyphenyl, 2,4-, 3,5- and 2,6-diethoxyphenyl, 2,4,6-triethoxyphenyl, 2-, 3- and 4-propoxyphenyl, 2,4-, 3,5- and 2,6-dipropoxyphenyl, 2-, 3- and 4-isopropoxyphenyl, 2,4- and 2,6-diisopropoxyphenyl and 2-, 3- and 4-butoxyphenyl; 2-, 3- and 4-chlorophenyl and 2,4-, 3,5- and 2,6-dichlorophenyl; 2-, 3- and 4-hydroxyphenyl and 2,4-, 3,5- and 2,6-dihydroxyphenyl; 2-, 3- and 4-cyanophenyl; 3- and 4-carboxyphenyl; 3- and 4-carboxamidophenyl, 3- and 4-N-methylcarboxamidophenyl and 3- and 4-N-ethylcarboxamidophenyl; 3- and 4-acetylaminophenyl, 3- and 4-propionylaminophenyl and 3- and 4-butyrylaminophenyl; 3- and 4-N-phenylaminophenyl, 3- and 4-N-(o-tolyl)aminophenyl, 3- and 4-N-(m-tolyl)aminophenyl and 3- and 4-N-(p-tolyl)aminophenyl; 3- and 4-(2-pyridyl)aminophenyl, 3- and 4-(3-pyridyl)-aminophenyl, 3- and 4-(4-pyridyl)aminophenyl, 3- and 4-(2-pyrimidyl)aminophenyl and 4-(4-pyrimidyl)aminophenyl;
4-phenylazophenyl, 4-(1-naphthylazo)phenyl, 4-(2-naphthylazo)phenyl, 4-(4-naphthylazo)phenyl, 4-(2-pyridylazo)phenyl, 4-(3-pyridylazo)phenyl, 4-(4-pyridylazo)phenyl, 4-(2-pyrimidylazo)phenyl, 4-(4-pyrimidylazo)phenyl and 4-(5-pyrimidylazo)phenyl;
phenoxy, phenylthio, 2-naphthoxy, 2-naphthylthio, 2-, 3- and 4-pyridyloxy, 2-, 3- and 4-pyridylthio, 2-, 4- and 5-pyrimidyloxy and 2-, 4- and 5-pyrimidylthio;
ethynyl, 1- and 2-propynyl, 1-, 2- and 3-butynyl, 1-, 2-, 3- and 4-pentynyl, 3-methyl-1-butynyl, 1-, 2-, 3-, 4- and 5-hexynyl, 3- and 4-methyl-1-pentynyl, 3,3-dimethyl-1-butynyl, 1-heptynyl, 3-, 4- and 5-methyl-1-hexynyl, 3,3-, 3,4- and 4,4-dimethyl-1-pentynyl, 3-ethyl-1-pentynyl, 1-octynyl, 3-, 4-, 5- and 6-methyl-1-heptynyl, 3,3-, 3,4-, 3,5-, 4,4- and 4,5-dimethyl-1-hexynyl, 3-, 4- and 5-ethyl-1-hexynyl, 3-ethyl-3-methyl-1-pentynyl, 3-ethyl-4-methyl-1-pentynyl, 3,3,4- and 3,4,4-trimethyl-1-pentynyl, 1-nonynyl, 1-decynyl, 1-undecynyl and 1-dodecynyl;
4-cyano-1-butynyl, 5-cyano-1-pentynyl, 6-cyano-1-hexynyl, 7-cyano-1-heptynyl and 8-cyano-1-octynyl;
4-hydroxy-1-butynyl, 5-hydroxy-1-pentynyl, 6-hydroxy-1-hexynyl, 7-hydroxy-1-heptynyl, 8-hydroxy-1-octynyl, 9-hydroxy-1-nonynyl, 10-hydroxy-1-decynyl, 11-hydroxy-1-undecynyl and 12-hydroxy-1-dodecynyl;
4-carboxy-1-butynyl, 5-carboxy-1-pentynyl, 6-carboxy-1-hexynyl, 7-carboxy-1-heptynyl, 8-carboxy-1-octynyl, 4-methylcarboxy-1-butynyl, 5-methylcarboxy-1-pentynyl, 6-methylcarboxy-1-hexynyl, 7-methylcarboxy-1-heptynyl, 8-methylcarboxy-1-octynyl, 4-ethylcarboxy-1-butynyl, 5-ethylcarboxy-1-pentynyl, 6-ethylcarboxy-1-hexynyl, 7-ethyl-carboxy-1-heptynyl and 8-ethylcarboxy-1-octynyl;
1-ethenyl, 1- and 2-propenyl, 1-, 2- and 3-butenyl, 1-, 2-, 3- and 4-pentenyl, 3-methyl-1-butenyl, 1-, 2-, 3-, 4- and 5-hexenyl, 3- and 4-methyl-1-pentenyl, 3,3-dimethyl-1-butenyl, 1-heptenyl, 3-, 4- and 5-methyl-1-hexenyl, 3,3-, 3,4- and 4,4-dimethyl-1-pentenyl, 3-ethyl-1-pentenyl, 1-octenyl, 3-, 4-, 5- and 6-methyl-1-heptenyl, 3,3-, 3,4-, 3,5-, 4,4- and 4,5-dimethyl-1-hexenyl, 3-, 4- and 5-ethyl-1-hexenyl, 3-ethyl-3-methyl-1-pentenyl, 3-ethyl-4-methyl-1-pentenyl, 3,3,4- and 3,4,4-trimethyl-1-pentenyl, 1-nonenyl, 1-decenyl, 1-undecenyl and 1-dodecenyl;
4-cyano-1-butenyl, 5-cyano-1-pentenyl, 6-cyano-1-hexenyl, 7-cyano-1-heptenyl and 8-cyano-1-octenyl;
4-hydroxy-1-butenyl, 5-hydroxy-1-pentenyl, 6-hydroxy-1-hexenyl, 7-hydroxy-1-heptenyl, 8-hydroxy-1-octenyl, 9-hydroxy-1-nonenyl, 10-hydroxy-1-decenyl, 11-hydroxy-1-undecenyl and 12-hydroxy-1-dodecenyl;
4-carboxy-1-butenyl, 5-carboxy-1-pentenyl, 6-carboxy-1-hexenyl, 7-carboxy-1-heptenyl, 8-carboxy-1-octenyl, 4-methylcarboxy-1-butenyl, 5-methylcarboxy-1-pentenyl, 6-methylcarboxy-1-hexenyl, 7-methylcarboxy-1-heptenyl, 8-methylcarboxy-1-octenyl, 4-ethylcarboxy-1-butenyl, 5-ethylcarboxy-1-pentenyl, 6-ethylcarboxy-1-hexenyl, 7-ethylcarboxy-1-heptenyl and 8-ethylcarboxy-1-octenyl.

Inventive naphthalene derivatives preferably bear 2× substituents.

Inventive perylene derivatives preferably bear 2× substituents.

Inventive terrylene derivatives preferably bear 2 or 4× substituents.

Inventive quaterrylene derivatives preferably bear 2, 4 or 6× substituents.

When they are prepared, the rylene derivatives (I) are generally obtained in the form of product mixtures with different degrees of substitution or as isomers which, if desired, can be separated, for example by chromatography.

In a preferred embodiment, the rylene derivatives (I) are used in the form of the mixture formed in the synthesis.

The symbols and indices in the formula (I) are preferably each defined as follows:

n is preferably 0, 1, 2 or 3.

m is preferably an integer from 0 to 2, i.e. 0.1 or 2, when n=0.

m is preferably an integer from 0 to 4, i.e. 0, 1, 2, 3 or 4, when n=1.

m is preferably an integer from 0 to 4, i.e. 0, 1, 2, 3 or 4, when n=2.

m is preferably an integer from 0 to 6, i.e. 0, 1, 2, 3 or 4, when n=3.

X is preferably the same or different and is, i.e. 0, 1, 2, 3, 4, 5 or 6,
C₁-C₂₀-alkyl, C₃-C₂₀-alken-2-yl or C₃-C₂₀-alkyn-2-yl whose alkyl chain may in each case be interrupted by one or more —O—, —S—, —NR¹—, —CO— and/or —SO₂— moieties and which may be mono- or polysubstituted by cyano, C₁-C₆-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic;
aryloxy, arylthio, hetaryloxy or hetarylthio, to each of which may be fused further saturated or unsaturated 5- to 7-membered rings whose carbon skeleton may be interrupted by one or more —O—, —S—, —NR¹—, —N═CR¹—, —CO—, —SO— and/or —SO₂— moieties, where the entire ring system may be mono- or polysubstituted by the alkyl radicals (1), cycloalkyl radicals (2), aryl or hetaryl radicals (3) mentioned for R and/or the (i) and/or (iv) radicals mentioned there,
or
a 5- to 9-membered ring which is bonded via a nitrogen atom and whose carbon chain may be interrupted by one or more —O—, —S—, —NR¹—, —CO— and/or —SO₂— moieties, to which may be fused one or two unsaturated or saturated 4- to 8-membered rings whose carbon chain may likewise be interrupted by these moieties and/or —N═, where the entire ring system may be mono- or polysubstituted by:
hydroxyl, nitro, —NHR², carboxyl, —COOR², —CONR²R³ or —NR²COR³;
C₁-C₃₀-alkyl whose carbon chain may be interrupted by one or more —O—, —S—, —NR¹—, —CO— and/or —SO₂— moieties and which may be mono- or polysubstituted by cyano, hydroxyl, nitro, C₁-C₆-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic;
C₅-C₈-cycloalkyl whose carbon skeleton may be interrupted by one or more —O—, —S— and/or —NR¹— moieties and/or which may be mono- or polysubstituted by C₁-C₆-alkyl;
aryl or hetaryl, each of which may be mono- or polysubstituted by C₁-C₁₈-alkyl, C₁-C₆-alkoxy, cyano, nitro, halogen, —CONR²R³, —NR²COR³, —SO₂NR²R³ and/or aryl- or hetarylazo, each of which may be substituted by C₁-C₁₀-alkyl, C₁-C₆-alkoxy or cyano,
or
C₁-C₁₂-alkoxy or NR³R⁴.

R¹ is preferably hydrogen or C₁-C₆-alkyl.

R², R³, R⁴ are preferably the same or different and are each hydrogen,
C₁-C₁₈-alkyl which may be substituted by C₁-C₆-alkoxy, halogen, hydroxyl, carboxyl and/or cyano; aryl or hetaryl which may be substituted by the above radicals mentioned for alkyl and by C₁-C₆-alkyl.

R is preferably C₁-C₃₀-alkyl whose carbon chain may be interrupted by one or more —O—, —S—, —NR¹—, —CO— and/or —SO₂— moieties and which may be mono- or polysubstituted by cyano, C₁-C₆-alkoxy, aryl which may be substituted by C₁-C₁₈-alkyl or C₁-C₆-alkoxy, and/or a 5- to 7-membered heterocyclic radical which is bonded via a nitrogen atom and which may comprise further heteroatoms and be aromatic;
C₅-C₈-cycloalkyl whose carbon skeleton may be interrupted by one or more —O—, —S— and/or —NR¹— moieties and/or which may be mono- or polysubstituted by C₁-C₆-alkyl;
aryl or hetaryl which may be mono- or polysubstituted by C₁-C₁₈-alkyl, C₁-C₆-alkoxy, cyano, nitro, halogen, —CONR²R³, —SO₂NR²R³, —COOR², —SO₃R² and/or aryl- or hetarylazo, each of which may be substituted by C₁-C₁₀-alkyl, C₁-C₆-alkoxy or cyano.

Preference is given to compounds of the formula (I) in which all symbols and indices each have the preferred definitions.

More preferably, the symbols and indices in the formula (I) are each defined as follows:

n is more preferably 0, 1, 2 or 3.

m is more preferably 0 or 2 when n=0.

m is more preferably 0, 2 or 4 when n=1.

m is more preferably 0, 2 or 4 when n=2.

m is more preferably 0, 2, 4 or 6 when n=3.

X is more preferably the same or different, preferably the same, and is NHR³, phenyloxy, phenylthio, phenylamino, pyrimidyloxy, pyrimidylthio, pyrrolidinyl or piperidinyl, in each case unsubstituted or substituted by one or more radicals from the group of (C₁-C₁₀)-alkyl, (C₁-C₁₀)-alkoxy, cyano, COOR¹ and SO₃R¹.

R¹ is more preferably hydrogen or C₁-C₆-alkyl.

R³ is more preferably hydrogen, C₁-C₁₈-alkyl which may be substituted by C₁-C₆-alkoxy, halogen, hydroxyl, carboxyl and/or cyano; aryl or hetaryl which may be substituted by the above radicals mentioned for alkyl and by C₁-C₆-alkyl.

R is more preferably (C₄-C₂₀)-alkyl or phenyl which is unsubstituted or substituted by one or more (C₁-C₁₀)-alkyl groups.

Particular preference is given to compounds of the formula (I) in which all symbols and indices have the particularly preferred definitions.

Even more preferably, the symbols and indices in the formula (I) are each defined as follows:

n is even more preferably 0, 1, 2 or 3.

m is more preferably 0 or 2 when n=0.

m is more preferably 0, 2 or 4 when n=1.

m is more preferably 0, 2 or 4 when n=2.

m is more preferably 0, 2, 4 or 6 when n=3.

X is even more preferably the same or different, preferably the same, and is NH(C₁-C₁₀)-alkyl, phenoxy, phenylamino, pyrrolidinyl, piperidinyl, where the radicals mentioned are unsubstituted or each substituted by from one to three (C₁-C₈)-alkyl groups.

R is even more preferably (C₄-C₂₀)-alkyl or phenyl which is substituted by one or more (C₁-C₈)-alkyl groups.

Very particular preference is given to compounds of the formula (I) in which all symbols and indices have the very particularly preferred definitions.

Especially preferably, the symbols and indices in the formula (I) are each defined as follows:

n is especially preferably 0, 1, 2 or 3.

m is especially preferably 0 or 2 when n=0.

m is especially preferably 0, 2 or 4 when n=1.

m is especially preferably 0, 2 or 4 when n=2.

m is especially preferably 0, 2, 4 or 6 when n=3.

X is especially preferably

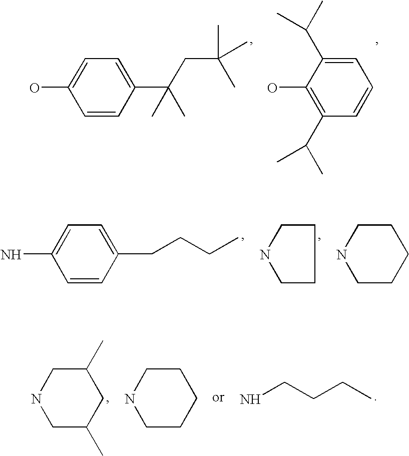

and

R is especially preferably

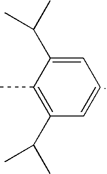

Special preference is given to compounds of the formula (I) in which all symbols and indices have the especially preferred definitions.

Preferred compounds of the formula (I) are also those of the formula (IA)

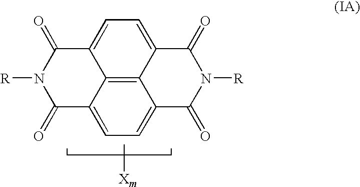

where the symbols and indices are each as defined in the formula (I).

Particularly preferred compounds of the formula (I) are those of the formula (IAa)

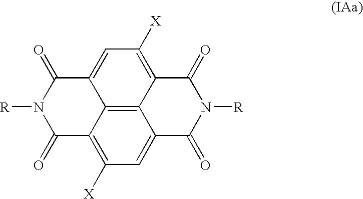

where the symbols and indices are each as defined in the formula (I).

Preference is further given to the compounds of the formula (IB)

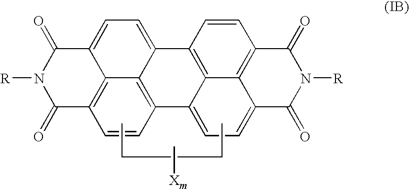

where the symbols and indices are each defined as follows:

X is the same or different and is C₁-C₁₂-alkoxy, aryloxy, if appropriate substituted as specified in formula (I), or a 5- to 9-membered ring bonded via a nitrogen atom, if appropriate modified as specified in formula (I); and

R, m are each as defined in the formula (I).

Compounds of the formula (IA) and (IB) are described, for example, in EP-A 0 648 817 and WO 97/22607.

Preference is further given to compounds of the formula (IC)

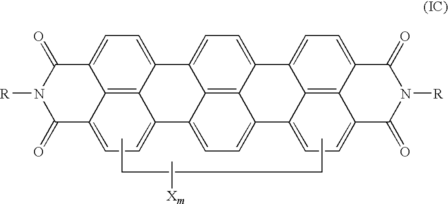

where X is aryloxy, if appropriate substituted as specified in formula (I), and m and R are each as defined in the formula (I).

Preferred compounds of the formula (I) are those of the formula (ID)

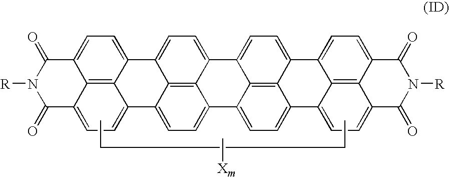

where X is phenoxy, if appropriate substituted as specified in formula (I), and m and R are each as defined in the formula (I).

Preferred compounds of the formula (ID) are those of the formula (IDd)

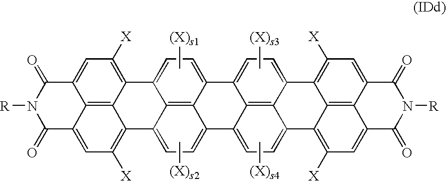

where s1, s2, s3, s4=0 or 1

and s1+s2+s3+s4=2,

where the symbols and indices are each as defined in the formula (ID).

Compounds of the formula (ID) are described, for example, in EP-A 0 596 292 and WO 96/22332.

A further preferred group of compounds of the formula (I) is that of those in which at least one X group is a radical of the formula

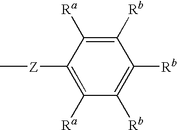

where the symbols are defined as follows:

Z is sulfur or oxygen;

R¹ are the same or different and are each:
(i) C₁-C₃₀-alkyl which does not comprise a tertiary carbon atom in the 1-position and whose carbon chain may be interrupted by one or more —O—, —S—, —NR^{c}—, —N═CR^{c}—, —C≡C—, —CR^{c}═CR^{c}—, —CO—, —SO— and/or —SO₂— moieties and which may be mono- or polysubstituted by: C₁-C₁₂-alkoxy, C₁-C₆-alkylthio, —C≡CR^{c}, —CR^{c}═CR^{c}₂, hydroxyl, mercapto, halogen, cyano, nitro, —NR^{d}R^{e}, —NR^{d}COR^{e}, —CONR^{d}R^{e}, —SO₂NR^{d}R^{e}, —COOR^{d}, —SO₃R^{d}, saturated or unsaturated C₄-C₇-cycloalkyl whose carbon skeleton may be interrupted by one or more —O—, —S—, —NR^{c}—, —N═CR^{c}—, —CR^{c}═CR^{c}—, —CO—, —SO— and/or —SO₂— moieties, and/or aryl, where aryl and cycloalkyl may be mono- or polysubstituted by C₁-C₁₈-alkyl and/or the above radicals mentioned as substituents for alkyl;
(ii) C₃-C₈-cycloalkyl which does not comprise a tertiary carbon atom in the 1-position and whose carbon skeleton may be interrupted by one or more —O—, —S—, —NR^{c}—, —N═CR^{c}—, CR^{c}—, —CO—, —SO— and/or —SO₂ moieties and to which may be fused further saturated or unsaturated 5- to 7-membered rings whose carbon skeleton may be interrupted by one or more —O—, —S—, —NR^{c}—, —N═CR^{c}—, —CR^{c}═CR^{c}, —CO—, —SO— and/or —SO₂— moieties, where the entire ring system may be mono- or polysubstituted by: C₁-C₁₈-alkyl, C₁-C₁₂-alkoxy, C₁-C₆-alkylthio, —C≡CR^{c}, —CR^{c}═CR^{c}₂, hydroxyl, mercapto, halogen, cyano, nitro, —NR^{d}R^{e}, —NR^{d}COR^{e}, —CONR^{d}R^{e}, —SO₂NR^{d}R^{e} and/or —COOR^{d};
(iii) aryl or hetaryl, to each of which may be fused further saturated or unsaturated 5- to 7-membered rings whose carbon skeleton may be interrupted by one or more —O—, —S—, —NR^{c}—, —N═CR^{c}—, —CR^{c}═CR^{c}—, —CO—, —SO— and/or —SO₂— moieties, where the entire ring system may be mono- or polysubstituted by: C₁-C₁₈-alkyl, C₁-C₁₂-alkoxy, C₁-C₆-alkylthio, —C≡CR^{c}, —CR^{c}═CR^{c}₂, hydroxyl, mercapto, halogen, cyano, nitro, —NR^{d}R^{e}, —NR^{d}COR^{e}, —CONR^{d}R^{e}, —SO₂NR^{d}R^{e}, —COOR^{d}, —SO₃R^{d}, aryl and/or hetaryl, each of which may be substituted by C₁-C₁₈-alkyl, C₁-C₁₂-alkoxy, hydroxyl, mercapto, halogen, cyano, nitro, —NR^{d}R^{e}, —NR^{d}COR^{e}, —CONR^{d}R^{e}, —SO₂NR^{d}R^{e}, —COOR^{d} and/or —SO₃R^{d};
(iv) a —U-aryl radical which may be mono- or polysubstituted by the above radicals specified as substituents for the aryl radicals (iii), where U is an —O—, —S—, —NR^{c}—, —CO—, —SO— or —SO₂— moiety;
(v) C₁-C₁₂-alkoxy, C₁-C₆-alkylthio, —C≡CR^{c}, —CR^{c}═CR^{c}₂, hydroxyl, mercapto, halogen, cyano, nitro, —NR^{d}R^{e}, —NR^{d}COR^{e}, —CONR^{d}R^{e}, —SO₂NR^{d}R^{e}, —COOR^{d} or —SO₃R^{d};

R^{b} are the same or different and are each:
hydrogen;
one of the (i), (ii), (iii), (iv) and (v) radicals specified for R, where the alkyl radicals (i) and the cycloalkyl radicals (ii) may comprise a tertiary carbon atom in the 1-position;

R^{c} are the same or different and are each
hydrogen or C₁-C₁₈-alkyl;

R^{d}, R^{e} are the same or different and are each hydrogen;
C₁-C₁₈-alkyl whose carbon chain may be interrupted by one or more —O—, —S—, —CO— and/or —SO₂— moieties and which may be mono- or polysubstituted by C₁-C₁₂-alkoxy, C₁-C₆-alkylthio, hydroxyl, mercapto, halogen, cyano, nitro and/or —COOR^{c};
aryl or hetaryl, to each of which may be fused further saturated or unsaturated 5- to 7-membered rings whose carbon skeleton may be interrupted by one or more —O—, —S—, —CO— and/or —SO₂— moieties, where the entire ring system may be mono- or polysubstituted by C₁-C₁₂-alkyl and/or the above radicals specified as substituents for alkyl.

Compounds of the formula (I) which comprise corresponding X radicals are described in WO 2007/006717.

As a further preferred compound class, compounds of the formula (I) are used in which at least one X radical is defined as follows:

a 5- to 9-membered, preferably 5- to 7-membered, particularly preferred 5- to 6-membered ring which is bonded via a nitrogen atom and whose carbon chain may be interrupted by one or more —O—, —S—, —NR¹—, —CO— and/or —SO₂— moieties, to which may be fused one or two unsaturated or saturated 4- to 8-membered rings whose carbon chain may likewise be interrupted by these moieties and/or —N═, where the entire ring system may be mono- or polysubstituted by:

hydroxyl, nitro, —NHR², carboxyl, —COOR², —CONR²R³ and/or —NR²COR³; C₁-C₃₀-alkyl whose carbon chain may be interrupted by one or more —O—, —S—, —NR¹—, —CO— and/or —SO₂— moieties and which may be mono- or polysubstituted by cyano, hydroxyl, nitro, C₁-C₆-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic; C₅-C₈-cycloalkyl whose carbon skeleton may be interrupted by one or more —O—, —S— and/or —NR¹— moieties and/or which may be mono- or polysubstituted by C₁-C₆-alkyl; aryl or hetaryl, each of which may be mono- or polysubstituted by C₁-C₁₈-alkyl, C₁-C₆-alkoxy, cyano, nitro, halogen, —CONR²R³, —NR²COR³, —SO₂NR²R³ and/or aryl- or hetarylazo, each of which may be substituted by C₁-C₁₀-alkyl, C₁-C₆-alkoxy or cyano.

Cyclic amines from which these X groups are derived include in particular piperidines, pyrrolidines, piperazines, morpholines and thiomorpholines (1,4-thiazines), preference being given to the piperidines, pyrrolidines, piperazines and morpholines, and particular preference to the piperidines.

These cyclic amines may be chemically modified, i.e. their carbon chain may be interrupted not only by —O—, —S— or —NR¹—, but also by —CO—, —SO— or —SO₂—, they may have one or two aromatic or saturated 4- to 7-membered fused rings whose carbon chain may likewise be interrupted by the moieties mentioned, and they may be substituted by the alkyl, cycloalkyl and/or (het)aryl radicals mentioned at the outset. However, preference is given to the unmodified groups.

Examples of suitable amines include:
piperidine, 2 and 3-methylpiperidine, 6-ethylpiperidine, 2,6- and 3,5-dimethylpiperidine, 2,2,6,6-tetramethylpiperidine, 4-benzylpiperidine, 4-phenylpiperidine, piperidin-4-ol, piperidine-4-carboxylic acid, methyl piperidine-4-carboxylate, ethyl piperidine-4-carboxylate, piperidine-4-carboxamide, 2,2,6,6-tetramethylpiperidin-4-one, 2,2,6,6-tetramethylpiperidin-4-ylamine, decahydroquinoline and decahydroisoquinoline;
pyrrolidine, 2-methylpyrrolidine, 2,5-dimethylpyrrolidine, 2,5-diethylpyrrolidine, tropanol, methyl pyrrolidine-2-carboxylate, ethyl pyrrolidine-2-carboxylate, benzyl pyrrolidine-2-carboxylate, pyrrolidine-2-carboxamide, 2,2,5,5-tetramethylpyrrolidine-3-carboxylic acid, methyl 2,2,5,5-tetramethylpyrrolidine-3-carboxylate, ethyl 2,2,5,5-tetramethylpyrrolidine-3-carboxylate, benzyl 2,2,5,5-tetramethylpyrrolidine-3-carboxylate, pyrrolidin-3-ylamine, (2,6-dimethylphenyl)pyrrolidin-2-ylmethylamine, (2,6-diisopropylphenyl)pyrrolidin-2-ylmethylamine and dodecahydrocarbazole;
piperazine, diketopiperazine; 1-benzylpiperazine, 1-phenethylpiperazine, 1-cyclohexyl-piperazine, 1-phenylpiperazine, 1-(2,4-dimethylphenyl)piperazine, 1-(2-, 3- and 4-methoxyphenyl)piperazine, 1-(2-, 3- and 4-ethoxyphenyl)piperazine, 1-(2-, 3- and 4-fluorophenyl)piperazine, 1-(2-, 3- and 4-chlorophenyl)piperazine, 1-(2-, 3- and 4-bromophenyl)piperazine, 1-, 2- and 3-pyridin-2-ylpiperazine and 1-benzo[1,3]dioxol-4-ylmethylpiperazine;
morpholine, 2,6-dimethylmorpholine, 3,3,5,5-tetramethylmorpholine, morpholin-2- and -3-ylmethanol, morpholin-2- and -3-ylacetic acid, methyl morpholin-2- and -3-ylacetate, ethyl morpholin-2- and -3-ylacetate, methyl 3-morpholin-3-ylpropionate, ethyl 3-morpholin-3-ylpropionate, tert-butyl 3-morpholin-3-ylpropionate, morpholin-2- and -3-ylacetamide, 3-morpholin-3-ylpropionamide, 3-benzylmorpholine, 3-methyl-2-phenyl-morpholine, 2- and 3-phenylmorpholine, 2-(4-methoxyphenyl)morpholine, 2-(4-tri-fluoromethylphenyl)morpholine, 2-(4-chlorophenyl)morpholine, 2-(3,5-dichlorophenyl)-morpholine, morpholine-2- and -3-carboxylic acid, methyl morpholine-3-carboxylate, 3-pyridin-3-ylmorpholine, 5-phenylmorpholin-2-one, 2-morpholin-2-ylethylamine and phenoxazine;
thiomorpholine, 2- and 3-phenylthiomorpholine, 2- and 3-(4-methoxyphenyl)thiomorpholine, 2- and 3-(4-fluorophenyl)thiomorpholine, 2- and 3-(4-trifluoromethyl-phenyl)thiomorpholine, 2- and 3-(2-chlorophenyl)thiomorpholine, 4-(2-aminoethyl)thiomorpholine, 3-pyridin-3-ylthiomorpholine, 3-thiomorpholinecarboxylic acid, 6,6-dimethyl-5-oxo-3-thiomorpholinecarboxylic acid, 3-thiomorpholinone and 2-phenylthiomorpholin-3-one, and also the thiomorpholine oxides and dioxides.

Compounds of the formula (I) which comprise corresponding X radicals are described in WO 2006/058674.

Equally preferred are quaterrylene compounds (n=3) in which m is equal to 0 (i.e. the quaterrylene skeleton is unsubstituted) and the R radicals are each independently C₈-C₁₈-alkyl or phenyl which is disubstituted by C₁-C₄-alkyl, and secondly those in which m assumes a value of from 2 to 6, X is defined as phenoxy, phenylthio, pyrimidyloxy or pyrimidylthio, each of which may be substituted by C₁-C₄-alkyl, the R radicals are each C₅-C₈-cycloalkyl or phenyl, pyridyl or pyrimidyl, each of which is mono- or polysubstituted by C₁-C₄-alkoxy, —CONHR¹ or —NHCOR¹, and R¹ is C₁-C₄-alkyl or phenyl which may be substituted by C₁-C₄-alkyl or C₁-C₄-alkoxy.

Further compounds of this class in which n is equal to 1, m assumes a value of 2 and X corresponds to identical aryloxy, arylthio, hetaryloxy or hetarylthio which are present in the 1,7-position of the perylene skeleton and are further substituted if appropriate, and which are suitable for the inventive use, are detailed in WO 97/22607.

More preferably, also taking account of the preferences mentioned above, the compounds used from this compound class in accordance with the invention are those compounds in which both R radicals in the formula (I) are the same.

A further preferred compound class used is that of compounds of the formula (I) in which the symbols and indices are defined as follows:

n is 0, 1, 2 or 3,

m is an integer from 0 to 2, i.e. 0, 1 or 2, when n=0,

m is an integer from 0 to 4, i.e. 0, 1, 2, 3 or 4, when n=1,

m is an integer from 0 to 4, i.e. 0, 1, 2, 3 or 4, when n=2,

m is an integer from 0 to 6, i.e. 0, 1, 2, 3, 4, 5 or 6, when n=3,

X is aryloxy, arylthio, hetaryloxy or hetarylthio, each of which may be mono- or polysubstituted by
C₁-C₃₀-alkyl whose carbon chain may be interrupted by one or more —O—, —S—, —NR¹—, —CO— and/or —SO₂— moieties, and/or which may be mono- or polysubstituted by hydroxyl, cyano, —COOR¹, —SO₃R¹ or C₁-C₆-alkoxy,
or
cyano, —COOR¹, —SO₃R¹ or C₁-C₆-alkoxy,
and

R is as defined in the formula (I).

Some of the compounds of the formula (I) are known and some are novel.

The invention therefore also provides compounds of the formula (I) from the group of:

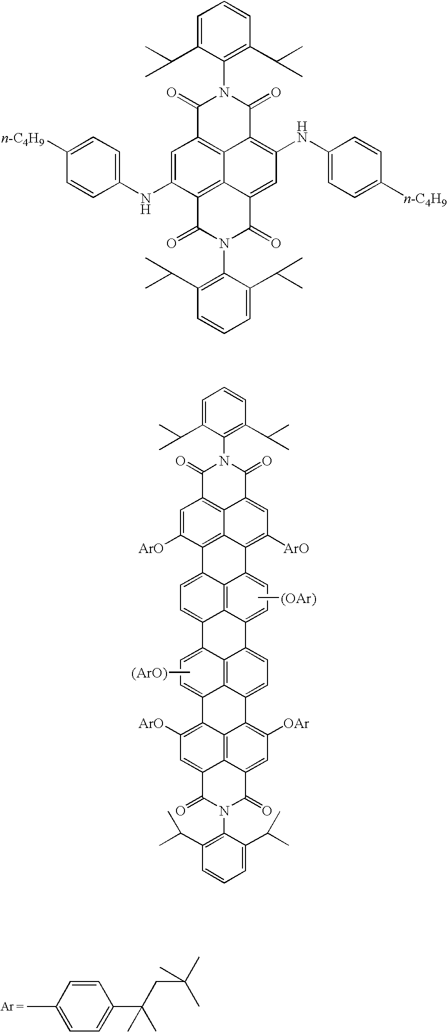

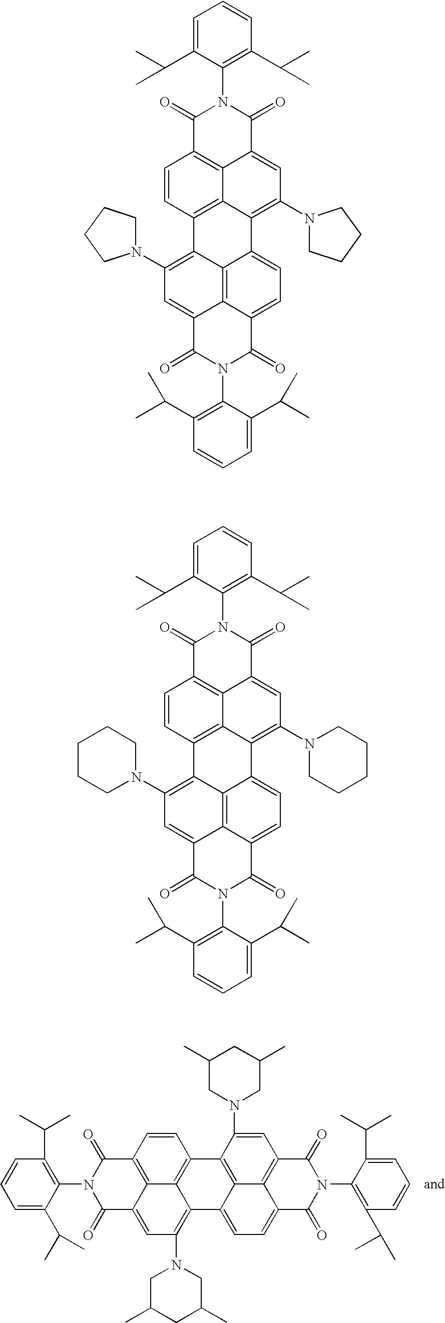

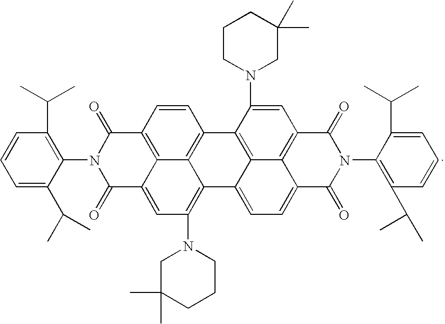

The compounds of the formulae (I) and (II) are prepared by known methods familiar to the person skilled in the art, as described, for example, in Houben Weyl, Methoden der organischen Chemie [Methods of organic chemistry], Thieme Verlag, Stuttgart, and the following documents: EP-A 0 596 292, EP-A 0 648 817, EP-A 0 657 436, WO 94/02570, WO 96/22331, WO 96/22332, WO 97/22607, WO 97/22608, WO 01/16109, WO 02/068538, WO 02/076988, DE-A 101 48 172, WO 2006/058674 and WO 2007/006717.

The present invention further provides liquids which comprise at least one compound of the formula (I) as a marker.

Useful liquids which can be marked with the compounds of the formula (I) preferably include oils such as mineral oils, vegetable and animal fatty oils, and ethereal oils.

Examples of such oils are natural oils such as olive oil, soybean oil or sunflower oil, or natural or synthetic motor oils, hydraulic oils or transmission oils, for example motor vehicle oil or sewing machine oil, or brake fluids and mineral oils which, according to the invention, comprise gasoline, kerosene, diesel oil and also heating oil.

Particular preference is given to mineral oils such as gasoline, kerosene, diesel oil or heating oil, in particular gasoline, diesel oil or heating oil.

Particularly advantageously, the abovementioned compounds of the formula (I) are used as markers for mineral oils in which labeling is simultaneously required, for example for tax reasons. In order to minimize the costs of labeling, but also in order to minimize possible interactions of the marked mineral oils with any other ingredients present, such as polyisobuteneamine (PIBA), efforts are made to minimize the amount of markers. A further reason to minimize the amount of markers may be to prevent their possible harmful influences, for example on the fuel intake and exhaust gas outlet region of internal combustion engines.

The compounds of the formula (I) to be used as markers are added to the liquids in such amounts that reliable detection is ensured. Typically, the (weight-based) total content of markers in the marked liquid is from about 0.1 to 5000 ppb, preferably from 1 to 2000 ppb and more preferably from 1 to 1000 ppb.

To mark the liquids, the compounds are generally added in the form of solutions (stock solutions). Especially in the case of mineral oils, suitable solvents for providing these stock solutions are preferably aromatic hydrocarbons such as toluene, xylene or relatively high-boiling aromatics mixtures.

In order to avoid too high a viscosity of such stock solutions (and hence poorer meterability and handling), a total concentration of the markers of from 0.5 to 50% by weight, based on the total weight of these stock solutions, is generally selected.

The compounds of the formula (I) may if appropriate also be used in a mixture with other markers/dyes as have been described, for example, at the outset. The total amount of the markers in the liquids is then typically within the range described above.

The present invention also provides a process for marking liquids, preferably oils, more preferably mineral oils, wherein a compound of the formula (I) is added to the liquid.

The compounds of the formula (I) are detected in the liquids by common methods. Since these compounds generally have a high absorption capacity and/or exhibit fluorescence, one possible example in the given case is spectroscopic detection.

The compounds of the formula (I) generally have their absorption maximum in the range from 600 to 1000 nm and/or fluorescence in the range from 600 to 1200 nm and can thus be detected easily with suitable instruments.

The detection can be effected in a manner known per se, for example by measuring the absorption spectrum of the liquids to be analyzed.

However, it is also possible to excite the fluorescence of the compounds of the formula (I) present in the liquids, advantageously with a semiconductor laser or a semiconductor diode. It is particularly favorable to employ a semiconductor laser or a semiconductor diode having a wavelength in the spectral range from λₘₐₓ−100 nm to λₘₐₓ+20 nm. λₘₐₓ is the wavelength of the absorption maximum of the marker. The wavelength of maximum emission is in the range from 620 to 1200 nm.

The fluorescence light thus generated is advantageously detected with a semiconductor detector, especially with a silicon photodiode or a germanium photodiode.

Detection is possible particularly advantageously when an interference filter and/or an edge filter (with a short-wavelength transmission edge in the range from λₘₐₓ to λₘₐₓ+80 nm) and/or a polarizer is disposed upstream of the detector.

By means of the abovementioned compounds, it is possible in a very simple manner to detect marked liquids, even when the compounds of the formula (I) are present only in a concentration of about 1 ppm (detection by absorption) or about 5 ppb (detection by fluorescence).

The invention also provides a process for identifying liquids which comprise a compound of the formula (I) in an amount which is sufficient to excite detectable fluorescence with radiation of a suitable wavelength, by
a) irradiating the liquid with electromagnetic radiation of a wavelength of from 600 to 1000 nm and
b) detecting the excited fluorescence radiation with an apparatus for detecting radiation in the near infrared range.

The rylene derivatives of the formula (I) can also be used as a component in additive concentrates (also referred to hereinafter, following the relevant terminology, as “packages”) which, in addition to a carrier oil and a mixture of various fuel additives, generally also comprise dyes and, for invisible fiscal or manufacturer-specific marking, additionally markers. These packages enable the supply of various mineral oil distributors from one “pool” of unadditized mineral oil and the imparting of the company-specific additization, color and marking to the mineral oil with the aid of their individual packages not until, for example, during the transfer to appropriate storage vessels.

The components present in such packages are then in particular:
a) at least one rylene derivative of the formula (I),
b) at least one carrier oil,
c) at least one additive selected from the group consisting of detergents, dispersants and valve seat wear-inhibiting additives,
d) and also, if appropriate, further additives and assistants.

The carrier oils used are typically viscous, high-boiling and in particular thermally stable liquids. They cover the hot metal surfaces, for example the intake valves, with a thin liquid film and thus prevent or delay the formation and deposition of decomposition products on the metal surfaces.

Carrier oils useful as component b) of the fuel and lubricant additive concentrates are, for example, mineral carrier oils (base oils), especially those of the Solvent Neutral (SN) 500 to 2000 viscosity class, synthetic carrier oils based on olefin polymers having M_{N}=from 400 to 1800, in particular based on polybutene or polyisobutene (hydrogenated or nonhydrogenated), on poly-alpha-olefins or poly(internal olefins) and also synthetic carrier oils based on alkoxylated long-chain alcohols or phenols. Adducts, to be used as carrier oils, of ethylene oxide, propylene oxide and/or butylene oxide to polybutyl alcohols or polyisobutene alcohols are described, for instance, in EP 277 345 A1; further polyalkene alcohol polyalkoxylates to be used are described in WO 00/50543 A1. Further carrier oils to be used also include polyalkene alcohol polyether amines, as detailed in WO 00/61708.

It is of course also possible to use mixtures of different carrier oils, as long as they are compatible with one another and with the remaining components of the packages.

Carburetors and intake systems of internal combustion engines, but also injection systems for fuel metering, are being contaminated to an increasing degree by impurities which are caused, for example, by dust particles from the air and uncombusted hydrocarbon residues from the combustion chamber.

To reduce or prevent these contaminations, additives (“detergents”) are added to the fuel to keep valves and carburetors or injection systems clean. Such detergents are generally used in combination with one or more carrier oils. The carrier oils exert an additional “wash function”, support and often promote the detergents in their action of cleaning and keeping clean, and can thus contribute to the reduction in the amount of detergents required.

It should also be mentioned here that many of the substances typically used as carrier oils display additional action as detergents and/or dispersants, which is why the proportion of the latter can be reduced in such a case. Such carrier oils having detergent/dispersant action are detailed, for instance, in the last-mentioned WO document.

It is also often impossible to clearly delimit the mode of action of detergents, dispersants and valve seat wear-inhibiting additives, which is why these compounds are listed in summary under component c). Customary detergents which find use in the packages are listed, for example, in WO 00/50543 A1 and WO 00/61708 A1 and include:
polyisobuteneamines which are obtainable according to EP-A 244 616 by hydro-formylation of highly reactive polyisobutene and subsequent reductive amination with ammonia, monoamines or polyamines, such as dimethyleneaminopropylamine, ethylenediamine, diethylenetriamine, triethylenetetramine or tetraethylenepentamine,
poly(iso)buteneamines which are obtainable by chlorination of polybutenes or polyisobutenes having double bonds predominantly in the β- and γ-position and subsequent amination with ammonia, monoamines or the abovementioned polyamines,
poly(iso)buteneamines which are obtainable by oxidation of double bonds in poly(iso)butenes with air or ozone to give carbonyl or carboxyl compounds and subsequent amination under reducing (hydrogenating) conditions,
polyisobuteneamines which are obtainable according to DE-A 196 20 262 from polyisobutene epoxides by reaction with amines and subsequent dehydration and reduction of the amino alcohols,
polyisobuteneamines which optionally comprise hydroxyl groups and are obtainable according to WO-A 97/03946 by reaction of polyisobutenes having an average degree of polymerization P of from 5 to 100 with nitrogen oxides or mixtures of nitrogen oxides and oxygen and subsequent hydrogenation of these reaction products,
polyisobuteneamines which comprise hydroxyl groups and are obtainable according to EP-A 476 485 by reaction of polyisobutene epoxides with ammonia, monoamines or the abovementioned polyamines,
polyetheramines which are obtainable by reaction of C₂-C₃₀-alkanols, C₆-C₃₀-alkanediols, mono- or di-C₂-C₃₀-alkylamines, C₁-C₃₀-alkylcyclohexanols or C₁-C₃₀-alkylphenols with from 1 to 30 mol of ethylene oxide and/or propylene oxide and/or butylene oxide per hydroxyl or amino group and subsequent reductive amination with ammonia, monoamines or the abovementioned polyamines, and also
“polyisobutene Mannich bases” which are obtainable according to EP-A 831 141 by reaction of polyisobutene-substituted phenols with aldehydes and monoamines or the abovementioned polyamines.

Further detergents and/or valve seat wear-inhibiting additives to be used are listed, for example, in WO 00/47698 A1 and comprise compounds which have at least one hydrophobic hydrocarbon radical having a number-average molecular weight (M_{N}) of from 85 to 20 000 and at least one polar moiety, and which are selected from:
(i) mono- or polyamino groups having up to 6 nitrogen atoms, of which at least one nitrogen atom has basic properties;
(ii) nitro groups, optionally in combination with hydroxyl groups;
(iii) hydroxyl groups in combination with mono- or polyamino groups, in which at least one nitrogen atom has basic properties;
(iv) carboxyl groups or their alkali metal or alkaline earth metal salts;
(v) sulfonic acid groups or their alkali metal or alkaline earth metal salts;
(vi) polyoxy-C₂-C₄-alkylene moieties which are terminated by hydroxyl groups, mono- or polyamino groups, in which at least one nitrogen atom has basic properties, or by carbamate groups;
(vii) carboxylic ester groups;
(viii) moieties derived from succinic anhydride and having hydroxyl and/or amino and/or amido and/or imido groups; and
(ix) moieties obtained by Mannich reaction of phenolic hydroxyl groups with aldehydes and mono- or polyamines.

Additives comprising mono- or polyamino groups (i) are preferably polyalkenemono- or polyalkenepolyamines based on polypropene or on highly reactive (i.e. having predominantly terminal double bonds, usually in the β- and γ-positions) or conventional (i.e. having predominantly internal double bonds) polybutene or polyisobutene having M_{N}=from 300 to 5000. Such additives based on highly reactive polyisobutene, which can be prepared from the polyisobutene (which may comprise up to 20% by weight of n-butene units) by hydroformylation and reductive amination with ammonia, monoamines or polyamines, such as dimethylaminopropylamine, ethylenediamine, diethylenetriamine, triethylenetetramine or tetraethylenepentamine, are disclosed in particular in EP 244 616 A2. When polybutene or polyisobutene having predominantly internal double bonds (usually in the β- and γ-positions) are used as starting materials in the preparation of the additives, a possible preparative route is by chlorination and subsequent amination or by oxidation of the double bond with air or ozone to give the carbonyl or carboxyl compound and subsequent amination under reductive (hydrogenating) conditions. The amines used here for the amination may be the same as those used above for the reductive amination of the hydroformylated highly reactive polyisobutene. Corresponding additives based on polypropene are described in particular in WO 94/24231 A1.

Further preferred additives comprising monoamino groups (i) are the hydrogenation products of the reaction products of polyisobutenes having an average degree of polymerization P of from 5 to 100 with nitrogen oxides or mixtures of nitrogen oxides and oxygen, as described in particular in WO 97/03946 A1.

Further preferred additives comprising monoamino groups (i) are the compounds obtainable from polyisobutene epoxides by reaction with amines and subsequent dehydration and reduction of the amino alcohols, as described in particular in DE 196 20 262 A1.

Additives comprising nitro groups (ii), if appropriate in combination with hydroxyl groups, are preferably reaction products of polyisobutenes having an average degree of polymerization P of from 5 to 100 or from 10 to 100 with nitrogen oxides or mixtures of nitrogen oxides and oxygen, as described in particular in WO 96/03367 A1 and WO 96/03479 A1. These reaction products are generally mixtures of pure nitropolyisobutanes (e.g. α,β-dinitropolyisobutane) and mixed hydroxynitropolyisobutanes (e.g. α-nitro-β-hydroxypolyisobutane).

Additives comprising hydroxyl groups in combination with mono- or polyamino groups (iii) are in particular reaction products of polyisobutene epoxides obtainable from polyisobutene having preferably predominantly terminal double bonds and M_{N}=from 300 to 5000, with ammonia or mono- or polyamines, as described in particular in EP 476 485 A1.

Additives comprising carboxyl groups or their alkali metal or alkaline earth metal salts (iv) are preferably copolymers of C₂-C₄₀-olefins with maleic anhydride which have a total molar mass of from 500 to 20 000 and of whose carboxyl groups some or all have been converted to the alkali metal or alkaline earth metal salts and any remainder of the carboxyl groups has been reacted with alcohols or amines. Such additives are disclosed in particular by EP 307 815 A1. Such additives serve mainly to prevent valve seat wear and can, as described in WO 87/01126 A1, advantageously be used in combination with customary detergents such as poly(iso)buteneamines or polyetheramines.

Additives comprising sulfonic acid groups or their alkali metal or alkaline earth metal salts (v) are preferably alkali metal or alkaline earth metal salts of an alkyl sulfosuccinate, as described in particular in EP 639 632 A1. Such additives serve mainly to prevent valve seat wear and can be used advantageously in combination with customary detergents such as poly(iso)buteneamines or polyetheramines.

Additives comprising polyoxy-C₂-C₄-alkylene moieties (vi) are preferably polyethers or polyetheramines which are obtainable by reaction of C₂-C₆₀-alkanols, C₆-C₃₀-alkanediols, mono- or di-C₂-C₃₀-alkylamines, C₁-C₃₀-alkylcyclohexanols or C₁-C₃₀-alkylphenols with from 1 to 30 mol of ethylene oxide and/or propylene oxide and/or butylene oxide per hydroxyl group or amino group and, in the case of the polyetheramines, by subsequent reductive amination with ammonia, monoamines or polyamines. Such products are described in particular in EP 310 875 A1, EP 356 725 A1, EP 700 985 A1 and U.S. Pat. No. 4,877,416. In the case of polyethers, such products also have carrier oil properties. Typical examples of these are tridecanol butoxylates, isotridecanol butoxylates, isononylphenol butoxylates and polyisobutenol butoxylates and propoxylates and also the corresponding reaction products with ammonia.

Additives comprising carboxylic ester groups (vii) are preferably esters of mono-, di- or tricarboxylic acids with long-chain alkanols or polyols, in particular those having a minimum viscosity of 2 mm²/s at 100° C., as described in particular in DE 38 38 918 A1. The mono-, di- or tricarboxylic acids used may be aliphatic or aromatic acids, and particularly suitable ester alcohols or ester polyols are long-chain representatives having, for example, from 6 to 24 carbon atoms. Typical representatives of the esters are adipates, phthalates, isophthalates, terephthalates and trimellitates of isooctanol, of isononanol, of isodecanol and of isotridecanol. Such products also satisfy carrier oil properties.

Additives which comprise moieties derived from succinic anhydride and have hydroxyl and/or amino and/or amido and/or imido groups (viii) are preferably corresponding derivatives of polyisobutenylsuccinic anhydride which are obtainable by reacting conventional or highly reactive polyisobutene having M_{N}=from 300 to 5000 with maleic anhydride by a thermal route or via the chlorinated polyisobutene. Particular interest attaches to derivatives with aliphatic polyamines such as ethylenediamine, diethylenetriamine, triethylenetetramine or tetraethylenepentamine. Such gasoline fuel additives are described in particular in U.S. Pat. No. 4,849,572.

Additives comprising moieties obtained by Mannich reaction of phenolic hydroxyl groups with aldehydes and mono- or polyamines (ix) are preferably reaction products of polyisobutene-substituted phenols with formaldehyde and mono- or polyamines such as ethylenediamine, diethylenetriamine, triethylenetetramine, tetraethylenepentamine or dimethylaminopropylamine. The polyisobutenyl-substituted phenols may stem from conventional or highly reactive polyisobutene having M_{N}=from 300 to 5000. Such “polyisobutene-Mannich bases” are described in particular in EP 831 141 A1.

For a more precise definition of the additives detailed individually, reference is explicitly made here to the disclosures of the abovementioned prior art documents.

Dispersants as component c) are, for example, imides, amides, esters and ammonium and alkali metal salts of polyisobutenesuccinic anhydrides. These compounds find use especially in lubricant oils, but sometimes also as detergents in fuel compositions.

Further additives and assistants which may, if appropriate, be present as component d) of the packages are
organic solvents, for example alcohols such as methanol, ethanol, propanol, isopropanol, butanol, isobutanol, sec-butanol, pentanol, isopentanol, neopentanol or hexanol, for example glycols such as 1,2-ethylene glycol, 1,2- or 1,3-propylene glycol, 1,2-, 2,3- or 1,4-butylene glycol, di- or triethylene glycol or di- or tripropylene glycol, for example ethers such as methyl tert-butyl ether, 1,2-ethylene glycol monomethyl ether or 1,2-ethylene glycol dimethyl ether, 1,2-ethylene glycol monoethyl ether or 1,2-ethylene glycol diethyl ether, 3-methoxypropanol, 3-isopropoxypropanol, tetrahydrofuran or dioxane, for example ketones such as acetone, methyl ethyl ketone or diacetone alcohol, for example esters such as methyl acetate, ethyl acetate, propyl acetate or butyl acetate, for example lactams such as N-methylpyrrolidinone (NMP), for example aliphatic or aromatic hydrocarbons and also mixtures thereof such as pentane, hexane, heptane, octane, isooctane, petroleum ether, toluene, xylene, ethylbenzene, tetralin, decalin, dimethylnaphthalene or white spirit and, for example, mineral oil such as gasoline, kerosene, diesel oil or heating oil,
corrosion inhibitors, for example based on ammonium salts, having a tendency to form films, of organic carboxylic acids or of heterocyclic aromatics in the case of ferrous metal corrosion protection,
antioxidants or stabilizers, for example based on amines such as p-phenylenediamine, dicyclohexylamine or derivatives thereof or on phenols such as 2,4-di-tert-butylphenol or 3,5-di-tert-butyl-4-hydroxyphenylpropionic acid,
demulsifiers,
antistats,
metallocenes such as ferrocene or methylcyclopentadienylmanganese tricarbonyl,
lubricity improvers (lubricity additives) such as certain fatty acids, alkenylsuccinic esters, bis(hydroxyalkyl) fatty amines, hydroxyacetamides or castor oil,
amines for reducing the pH of the fuel,
further markers other than rylene derivatives of the formula (I) and their preferred embodiments and
dyes.

The concentration of component a), i.e. of the at least one rylene derivative of the formula (I) or preferred embodiments thereof, in the packages is typically selected in such a magnitude that, after addition of the package to the mineral oil, the desired concentration of marker(s) is present therein. Typical concentrations of the markers in the mineral oil are, for instance, in the range from 0.01 up to a few 10 s of ppm by weight.

Component b), i.e. the at least one carrier oil, is present in the packages typically in a concentration of from 1 to 50% by weight, in particular from 5 to 30% by weight, and component c), i.e. the at least one detergent and/or the at least one dispersant, typically in a concentration of from 25 to 90% by weight, in particular from 30 to 80% by weight, based in each case on the total amount of components a) to c) and, if appropriate, d), the sum of the individual concentrations of components a) to c) and, if appropriate, d) adding up to 100% by weight.

When, as component d), corrosion inhibitors, antioxidants or stabilizers, demulsifiers, antistats, metallocenes, lubricity improvers and amines to reduce the pH of the fuel are present in the packages, the sum of their concentrations typically does not exceed 10% by weight, based on the total amount of the package (i.e. the total amount of components a) to c) and d)), the concentration of the corrosion inhibitors and demulsifiers being typically in the range of from in each case about 0.01 to 0.5% by weight of the total amount of the package.

When, as component d), additional organic solvents (i.e. not already introduced with the remaining components) are present in the packages, the sum of their concentrations typically does not exceed 20% by weight, based on the total amount of the package. These solvents generally stem from solutions of the markers and/or dyes, which are added to the packages instead of the pure markers and/or dyes with a view to more precise meterability.

When, as component d), further markers other than rylene derivatives of the formula (I) or preferred embodiments thereof are present in the packages, their concentration is in turn based on the content that they are to have after addition of the packages in mineral oil. That which was stated for component a) applies mutatis mutandis.

When, as component d), dyes are present in the inventive packages, their concentration is typically, for instance, between 0.1 to 5% by weight, based on the total amount of the package.

The invention will be illustrated in detail by the examples.

### EXAMPLE 1

### N,N′-Bis(2,6-diisopropylphenyl)-2,6-di-n-butylaminonaphthalene-1,4,5,8-tetracarboximide

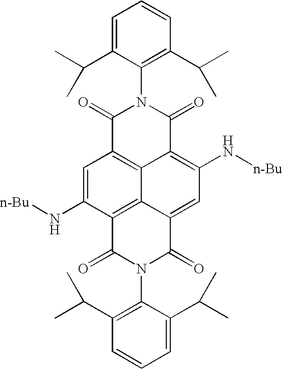

The preparation of the compound has been described by F. Würthner et al., Chem. Eur. J. 2002, 8, 4742 to 4750.

UV/Vis (toluene):

Absorption: λₘₐₓ (Ig ε)=346 (3.98), 362 (4.05), 616 nm (4.35)

Emission: λₘₐₓ=638 nm

### EXAMPLE 2

### N,N′-Bis(2,6-diisopropylphenyl)-2,6-di(4-n-butylanilino)naphthalene-1,4,5,8-tetracarboximide

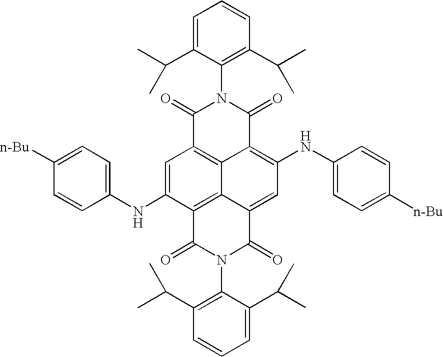

A solution of 4.5 g (30.2 mmol) of 4-n-butylaniline in 10 ml of xylene at 80° C. was added dropwise with stirring to a solution of 7.50 g (11.4 mmol) of N,N′-bis(2,6-diisopropylphenyl)-2,6-dichloronaphthalene-1,4,5,8-tetracarboximide (F. Würthner et al., Chem. Eur. J. 2002, 8, 4742 to 4750) in 30 ml of xylene. After stirring at 80° C. for 2 hours, the solution was heated to 125° C. and stirred at this temperature for 15 min. After cooling to room temperature, the solvent was evaporated on a rotary evaporator. The residue was admixed with 25.0 g (168 mmol) of 4-n-butylaniline and heated at 135° C. for 4 hours. The excess 4-n-butylaniline was then distilled off under reduced pressure (2 mbar). The residue was stirred up with 100 ml of glacial acetic acid. The precipitate was filtered off with suction, washed with water and dried at 50° C. in a vacuum drying cabinet. The crude product (8.7 g) was dissolved in 140 ml of toluene/cyclohexane mixture (4:1) and chromatographed on silica gel (70-200 μm). From a fraction which was homogeneous according to the thin-layer chromatogram, after removal of the solvent, 2.52 g (25% of theory) of dark blue powder with a melting point of >350° C. were obtained.

UV/Vis (toluene):

Absorption: λₘₐₓ (Ig ε)=316 (4.61), 370 (4.12), 616 nm (4.32)

Emission: λₘₐₓ=700 nm

### EXAMPLE 3

### N,N′-Bis(2,6-diisopropylphenyl)-2,6-dipyrrolidinonaphthalene-1,4,5,8-tetracarboximide

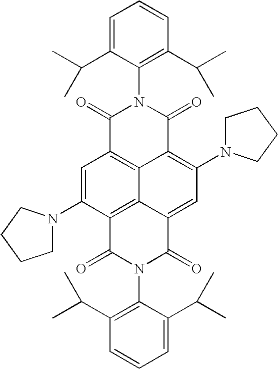

A solution of 3.93 g (6.0 mmol) of N,N′-bis(2,6-diisopropylphenyl)-2,6-dichloronaphthalene-1,4,5,8-tetracarboximide (F. Würthner et al., Chem. Eur. J. 2002, 8, 4742-4750), 0.90 g (12.8 mmol) of pyrrolidine and 1.2 g (12.0 mmol) of triethylamine in 75 ml of toluene was heated to boiling under reflux at 100° C. for 4 hours. After the addition of a further 0.90 g (12.7 mmol) of pyrrolidine, the solution was heated to boiling under reflux at 100° C. for 16.5 hours. The reaction solution was cooled to room temperature and filtered. The residue was washed with toluene and freed of solvent residues in a vacuum drying cabinet. The crude product (4.78 g) was chromatographed in methylene chloride/cyclohexane (9:1) on silica gel (70-200 μm). From a fraction which was homogeneous according to the thin-layer chromatogram, after removal of the solvent, 1.81 g (42% of theory) of dark blue powder having a melting point of >350° C. were obtained.

UV/Vis (toluene):

Absorption: λₘₐₓ (Ig ε)=300 (4.49), 348 (3.90), 364 (3.98), 554 (3.94), 596 nm (4.36)

Emission: λₘₐₓ=610 nm

### EXAMPLE 4

### N,N′-Bis(2,6-diisopropylphenyl)-1,7-dipyrrolidinoperylene-3,4,9,1 0-tetracarboximide

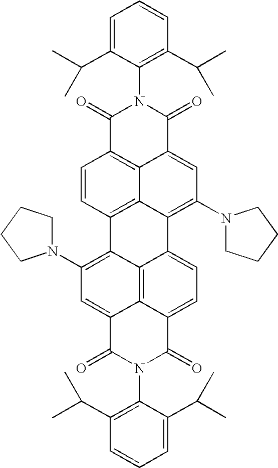

22.4 g (26.2 mmol) of N,N′-bis(2,6-diisopropylphenyl)-1,7-dibromoperylene-3,4,9,10-tetracarboximide (preparation as described in WO 97/22607), 5.54 g (77.9 mmol) of pyrrolidine and 13.82 g (100 mmol) of potassium carbonate were stirred at 60° C. in 100 ml of N-methylpyrrolidone (NMP) under nitrogen for 11.5 hours. After cooling to room temperature, 400 ml of water were added. The precipitate was filtered off with suction, washed with water and dried at 75° C. in a vacuum drying cabinet. The crude product (20.24 g) was heated in 100 ml of pyrrolidine under reflux (88° C.) to boiling for 7 hours. After cooling to room temperature, the reaction mixture was admixed with a solution of 80 ml of water and 20 ml of methanol. The precipitate was filtered off with suction, washed with 200 ml of 20% aqueous methanol then with 300 ml of water, and dried at 75° C. in a vacuum drying cabinet. The crude product thus obtained (17.29 g) was recrystallized from 173 ml of tetrahydronaphthalene. 3.91 g of green powder were obtained, which were dissolved in cyclohexane/ethyl acetate (3:1) and then purified on silica gel (60-200 nm). From a fraction which was homogeneous according to the thin-layer chromatogram, after removal of the solvent, 2.03 g (9% of theory) of green powder with a melting point of >350° C. were obtained.

UV/Vis (toluene):

Absorption: λₘₐₓ (Ig ε)=280 (4.41), 310 (4.44), 430 (4.24), 640 (4.36), 694 nm (4.65)

Emission: λₘₐₓ=711 nm

### EXAMPLE 5

### N,N′-Bis(2,6-diisopropylphenyl)-1,7-dipiperidinoperylene-3,4,9,1 0-tetracarboximide

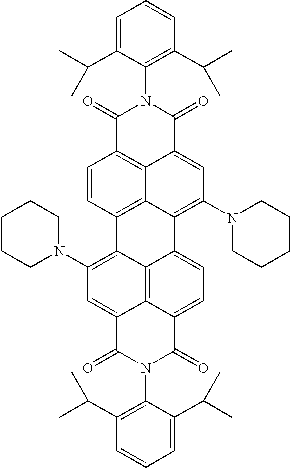

2.82 g (3.25 mmol) of N,N′-bis(2,6-diisopropylphenyl)-1,7-dibromoperylene-3,4,9,10-tetracarboximide (preparation as described in WO 97/22607) were stirred in 20 ml of pyrrolidine at room temperature for 6 hours. Subsequently, the reaction solution was heated to 60° C. and stirred at this temperature for 66 hours. After cooling to room temperature, the reaction solution was precipitated with water. The precipitate was filtered off with suction, washed with water and dried in a vacuum drying cabinet. The crude product (2.81 g) was dissolved in 50 ml of n-heptane/chloroform/acetone (55:40:5) and purified on silica gel (70-200 μm). From a fraction which was homogeneous according to the thin-layer chromatogram, after removal of the solvent, 1.16 g (41% of theory) of green powder with a melting point of >350° C. were obtained.

UV/Vis (toluene):

Absorption: λₘₐₓ (Ig ε)=280 (4.44), 410 (4.08), 432 (4.21), 676 nm (4.38)

Emission: λₘₐₓ=744 nm

### EXAMPLE 6

### N,N′-Bis(2,6-diisopropylphenyl)-1,7-bis(3,5-dimethylpiperidino)-perylene-3,4,9,1 0-tetracarboximide

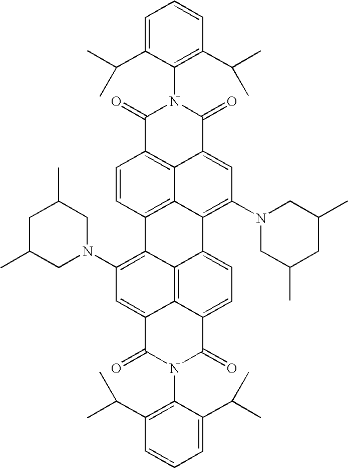

The preparation was effected analogously to that of Example 5.

UV/Vis (toluene):

Absorption: λₘₐₓ (Ig ε)=440 (4.14), 632 nm (4.27)

Emission: λₘₐₓ=738 nm

### EXAMPLE 7

### N,N′-Bis(2,6-diisopropylphenyl)-1,7-bis(3,3-dimethylpiperidino)-perylene-3,4,9, 1 0-tetracarboximide

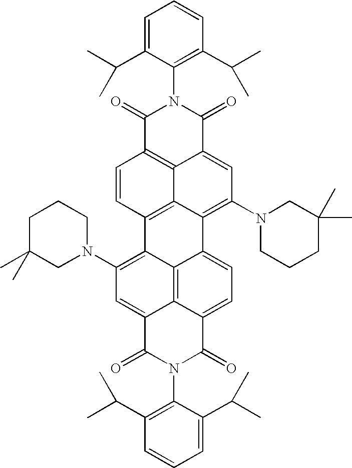

The preparation was effected analogously to that of Example 5.

UV/Vis (toluene):

Absorption: λₘₐₓ (Ig ε)=442 (4.13), 630 nm (4.26)

Emission: λₘₐₓ=740 nm

### EXAMPLE 8

### N,N′-Bis(2,6-diisopropylphenyl)-1,6,9,14-tetrakis(2,6-diisopropylphenoxy)terrylene-3,4,11,12-tetracarboximide

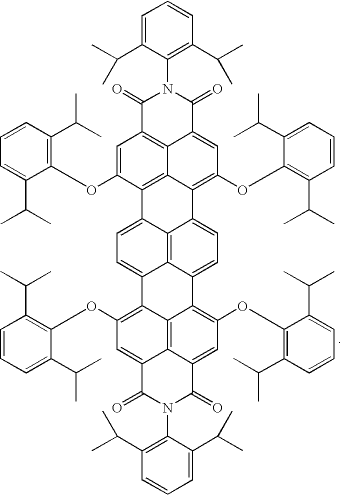

3.45 g (2.24 mmol) of N,N′-bis(2,6-diisopropylphenyl)-1,6,9,14-tetrabromoterrylene-3,4,11,12-tetracarboximide were initially charged in 197 ml of N-methylpyrrolidone (NMP), and 2.80 g (15,7 mmol) of 2,6-diisopropylphenol and 2.48 g (18.0 mmol) of potassium carbonate were added. The reaction mixture was stirred at 80° C. for 2 hours and at 85° C. for a further 5 hours. 50 ml of water were added dropwise to the reaction solution which was stirred overnight and then filtered. The residue was washed successively with 400 ml of 10% sulfuric acid, water and ethanol, and dried in a vacuum drying cabinet. 4.25 g of green solid were obtained, which were subjected to column chromatography on silica gel with toluene as the eluent for further purification.

UV/Vis (methylene chloride):

Absorption: λₘₐₓ (Ig ε)=694 nm (5.14)

Emission: λₘₐₓ=720 nm

### EXAMPLE 9

### N,N′-Bis(2,6-diisopropylphenyl)-1,6,7(8),11,16,17(18)-hexyl(4-tert-octylphenoxy)quaterrylene-3,4,13,14-tetracarboximide

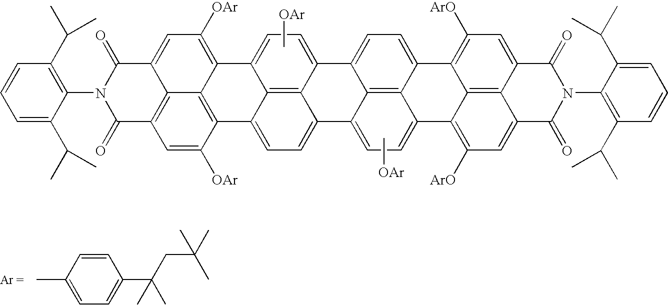

The preparation was effected analogously to that of Example 8.

UV/Vis (toluene):

Absorption: λₘₐₓ (Ig ε)=435 (4.15), 705 (4.84), 777 nm (5.12)

Emission: λₘₐₓ=814 nm

Storage Stability Test in Mineral Oils at 40° C.:

Approx. 50 mg of the particular compound were dissolved in 50 ml of Shellsol AB. Subsequently, the solutions were filtered through a fluted filter paper, and 6.5 ml or 5 ml of the filtrate were introduced into 10 ml ampules, made up to 10 ml with a commercial carrier oil or detergent based on polyisobuteneamine (PIBA) (solution with PIBA content of 50% by weight) (corresponding to a content of the particular compound of from 0.01 to 0.08%) and the solutions were stored in the ampules sealed air-tight at 40° C. in a water bath. After the storage times listed in the table below, samples were taken and analyzed in cuvettes having a diameter of 1 mm. In order to obtain better comparability of the storage tests, the table lists absorbances as a function of the particular storage time normalized to the reference, i.e. the starting absorbance of the unstored sample.

The tests demonstrate outstanding storage stability of the compounds used in accordance with the invention.

| | | | | Absorbance |
|---|---|---|---|---|
| | | Storage time | Normalized | maximum |
| Example | Additive | [h] | absorbance | [nm] |
| 1 | Detergent | 0 | 1.00 | 612 |
| | | 817 | 1.01 | |
| 1 | Carrier oil | 0 | 1.00 | 614 |
| | | 817 | 0.81 | |
| 2 | Detergent | 0 | 1.00 | 613 |
| | | 861 | 0.98 | |
| 2 | Carrier oil | 0 | 1.00 | 614 |
| | | 861 | 0.76 | |
| 4 | Detergent | 0 | 1.00 | 694 |
| | | 672 | 0.96 | |
| 4 | Carrier oil | 0 | 1.00 | 700 |
| | | 672 | 0.93 | |
| 6 | Detergent | 0 | 1.00 | 625 |
| | | 833 | 1.02 | |
| 6 | Carrier oil | 0 | 1.00 | 630 |
| | | 833 | 0.89 | |
| 7 | Detergent | 0 | 1.00 | 623 |
| | | 477 | 1.03 | |
| | | 833 | 1.03 | |
| 7 | Carrier oil | 0 | 1.00 | 631 |
| | | 477 | 0.95 | |
| | | 833 | 0.94 | |
| 8 | Detergent | 0 | 1.00 | 690 |
| | | 672 | 1.00 | |
| 8 | Carrier oil | 0 | 1.00 | 692 |
| | | 672 | 0.96 | |
| 9 | Detergent | 0 | 1.00 | 778 |
| | | 332 | 1.00 | |
| | | 818 | 0.99 | |
| 9 | Carrier oil | 0 | 1.00 | 780 |
| | | 332 | 0.93 | |
| | | 818 | 0.84 | |

1. A process for marking a mineral oil, comprising adding to the mineral oil a rylene derivative of formula (I) 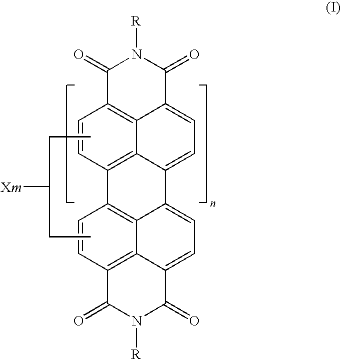
where the symbols and indices are defined as follows
n is an integer from 0 to 3;
m is an integer from 0 to 2 when n=0;
m is an integer from 0 to 4 when n=1;
m is an integer from 0 to 4 when n=2;
m is an integer from 0 to 6 when n=3;
X is the same or different and is
halogen
or
C₁-C₂₀-alkyl, C₃-C₂₀-alken-2-yl or C₃-C₂₀-alkyn-2-yl, whose alkyl chain may in each case be interrupted by one or more —O—, —S—, —CO— and/or —SO₂— moieties and which may be mono- or polysubstituted by cyano, C₁-C₆-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic;
or
aryloxy, arylthio, hetaryloxy or hetarylthio, to each of which may be fused further saturated or unsaturated 5- to 7-membered rings whose carbon skeleton may be interrupted by one or more —O—, —S—, —NR¹—, —N═CR¹—, —CO—, —SO— and/or —SO₂— moieties, where the entire ring system may be mono- or polysubstituted by the alkyl radicals (1), cycloalkyl radicals (2), aryl or hetaryl radicals (3) mentioned for R and/or the (i) and/or (iv) radicals mentioned there;
or
a 5- to 9-membered ring which is bonded via a nitrogen atom and whose carbon chain may be interrupted by one or more —O—, —S—, —NR¹—, —CO— and/or —SO₂— moieties, to which may be fused one or two unsaturated or saturated 4- to 8-membered rings whose carbon chain may likewise be interrupted by these moieties and/or —N═, where the entire ring system may be mono- or polysubstituted by: hydroxyl, nitro, —NHR², carboxyl, —COOR², —CONR²R³ or —NR²COR³;
C₁-C₃₀-alkyl whose carbon chain may be interrupted by one or more —O—, —S—, NR¹—, —CO— and/or —SO₂— moieties and which may be mono- or polysubstituted by cyano, hydroxyl, nitro, C₁-C₆-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic;
C₅-C₈-cycloalkyl whose carbon skeleton may be interrupted by one or more —O—, —S— and/or —NR¹— moieties and/or which may be mono- or polysubstituted by C₁-C₆-alkyl;
aryl or hetaryl, each of which may be mono- or polysubstituted by C1-C18-alkyl, C₁-C₆-alkoxy, cyano, nitro, halogen, —CONR²R³, —NR²COR³, —SO₂NR²R³ and/or aryl- or hetarylazo, each of which may be substituted by C₁-C₁₀-alkyl, C₁-C₆-alkoxy or cyano,
or
C₁-C₁₂-alkoxy, C₁-C₁₂-alkylthio or NR³R⁴;
R¹ is the same or different and is hydrogen or C₁-C₁₈-alkyl,
R², R³, R⁴ are the same or different and are each hydrogen,
C₁-C₁₈-alkyl whose carbon chain may be interrupted by one or more —O—, —S—, —CO—, —SO— and/or —SO₂— moieties and which may be mono- or polysubstituted by C₁-C₁₂-alkoxy, C₁-C₆-alkylthio, hydroxyl, mercapto, halogen, cyano, nitro and/or —COOR¹; aryl or hetaryl, to each of which may be fused further saturated or unsaturated 5- to 7-membered rings whose carbon skeleton may be interrupted by one or more —O—, —S—, —CO— and/or —SO₂— moieties, where the entire ring system may be mono- or polysubstituted by C₁-C₁₂-alkyl and/or the aforementioned radicals mentioned as substituents for alkyl;
and
R is the same or different and is hydrogen,
(1) C₁-C₃₀-alkyl whose carbon chain may be interrupted by one or more —O—, —S—, —NR¹—, —N═CR¹—, —C≡C—, —CR¹═CR¹—, —CO—, —SO— and/or —SO₂— moieties and which may be mono- or polysubstituted by:
(i) C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂N²R³, —COOR² and/or —SO₃R;
(ii) aryl or hetaryl, to which may be fused further saturated or unsaturated 5- to 7-membered rings whose carbon skeleton may be interrupted by one or more —O—, —S—, —NR¹—, —N═CR¹—, —CR¹═CR¹—, —CO—, —SO— and/or —SO₂— moieties, where the entire ring system may be mono- or polysubstituted by: C₁-C₁₈-alkyl, C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR², —SO₃R², aryl and/or hetaryl, each of which may be substituted by C₁-C₁₈-alkyl, C₁-C₁₂-alkoxy, hydroxyl, mercapto, halogen, cyano, nitro, —NR²R³, —NR²COR³, —CONR²R³, —SO₂NR²R³, —COOR² and/or —SO₃R²;
(iii) C₃-C₈-cycloalkyl, whose carbon skeleton may be interrupted by one or more —O—, —S—, —NR¹—, —N═CR¹—, —CR¹═CR¹—, —CO—, —SO— and/or —SO₂ moieties and to which may be fused further saturated or unsaturated 5- to 7-membered rings whose carbon skeleton may be interrupted by one or more —O—, —S—, —NR¹—, —N═CR¹—, —CR¹═CR¹—, —CO—, —SO— and/or —SO₂— moieties, where the entire ring system may be mono- or polysubstituted by: C₁-C₁₈-alkyl, C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, CONR²R³, —SO₂NR²R³, —COOR² and/or —SO₃R²;
(iv) a —U-aryl radical which may be mono- or polysubstituted by the above radicals mentioned as substituents for the aryl radicals (ii), where U is an —O—, —S—, —NR¹—, —CO—, —SO— or —SO₂— moiety;
(2) C₃-C₈-cycloalkyl to which may be fused further saturated or unsaturated 5- to 7-membered rings whose carbon skeleton may be interrupted by one or more —O—, —S—, —NR¹—, —N═CR¹—, —CR¹═CR¹—, —CO—, —SO— and/or —SO₂— moieties, where the entire ring system may be mono- or polysubstituted by: the (i), (ii), (iii), (iv) radicals and/or
(v) C₁-C₃₀-alkyl, whose carbon chain may be interrupted by one or more —O—, —S—, —NR¹—, —N═CR¹—, —C≡C—, —CR¹═CR¹—, —CO—, —SO— and/or —SO₂— moieties and which may be mono- or polysubstituted by: C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR², —SO₃R₂, aryl and/or saturated or unsaturated C₄-C₇-cycloalkyl whose carbon skeleton may be interrupted by one or more —O—, —S—, —NR¹—, —N═CR¹—, —CR¹═CR¹—, —CO—, —SO— and/or —SO₂— moieties, where the aryl and cycloalkyl radicals may each be mono- or polysubstituted by C₁-C₁₈-alkyl and/or the above radicals mentioned as substituents for alkyl;
(3) aryl or hetaryl, to which may be fused further saturated or unsaturated 5- to 7-membered rings whose carbon skeleton may be interrupted by one or more —O—, —S—, —N═CR¹—, —C≡C—, —CR¹═CR¹—, —CO—, —SO— and/or —SO₂— moieties, where the entire ring system may be substituted by the (i), (ii), (iii), (iv), (v) radicals and/or aryl- and/or hetarylazo, each of which may be substituted by C₁-C₁₀-alkyl, C₁-C₆-alkoxy and/or cyano.

2. The process according to claim 1, where the symbols and indices in formula (I) are each defined as follows:
n is 0, 1, 2 or 3;
m is an integer from 0 to 2 when n=0;
m is an integer from 0 to 4 when n=1;
m is an integer from 0 to 4 when n=2;
m is an integer from 0 to 6 when n=3;
X is the same or different and is
C₁-C₂₀-alkyl, C₃-C₂₀-alken-2-yl or C₃-C₂₀-alkyn-2-yl whose alkyl chain may in each case be interrupted by one or more —O—, —S—, —NR¹—, —CO— and/or —SO₂— moieties and which may be mono- or polysubstituted by cyano, C₁-C₆-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic;
aryloxy, arylthio, hetaryloxy or hetarylthio, to each of which may be fused further saturated or unsaturated 5- to 7-membered rings whose carbon skeleton may be interrupted by one or more —O—, —S—, —NR¹—, —N═CR¹—, —CO—, —SO— and/or —SO₂— moieties, where the entire ring system may be mono- or polysubstituted by the alkyl radicals (1), cycloalkyl radicals (2), aryl or hetaryl radicals (3) mentioned for R and/or the (i) and/or (iv) radicals mentioned there,
or
a 5- to 9-membered ring which is bonded via a nitrogen atom and whose carbon chain may be interrupted by one or more —O—, —S—, —NR¹—, —CO— and/or —SO₂— moieties, to which may be fused one or two unsaturated or saturated 4- to 8-membered rings whose carbon chain may likewise be interrupted by these moieties and/or —N═, where the entire ring system may be mono- or polysubstituted by:
hydroxyl, nitro, —NHR², carboxyl, —COOR², —CONR²R³ or —NR²COR³; C₁-C₃₀-alkyl whose carbon chain may be interrupted by one or more —O—, —S—, —NR¹—, —CO— and/or —SO₂— moieties and which may be mono- or polysubstituted by cyano, hydroxyl, nitro, C₁-C₈-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic;
C₅-C₈-cycloalkyl whose carbon skeleton may be interrupted by one or more —O—, —S— and/or —NR¹— moieties and/or which may be mono- or polysubstituted by C₁-C₆-alkyl;
aryl or hetaryl, each of which may be mono- or polysubstituted by C₁-C₁₈-alkyl, C₁-C₆-alkoxy, cyano, nitro, halogen, —CONR²R³, —NR²COR³, —SO₂NR²R³ and/or aryl- or hetarylazo, each of which may be substituted by C₁-C₁₀-alkyl, C₁-C₆-alkoxy or cyano,
or
C₁-C₁₂-alkoxy or NR³R⁴;
R¹ is hydrogen or C₁-C₆-alkyl;
R², R³, R⁴ are the same or different and are each hydrogen,
C₁-C₁₈-alkyl which may be substituted by C₁-C₆-alkoxy, halogen, hydroxyl, carboxyl and/or cyano;
aryl or hetaryl which may be substituted by the above radicals mentioned for alkyl and by C₁-C₆-alkyl; and
R is C₁-C₃₀-alkyl whose carbon chain may be interrupted by one or more —O—, S—, —NR¹—, —CO— and/or —SO₂— moieties and which may be mono- or polysubstituted by cyano, C₁-C₆-alkoxy, aryl which may be substituted by C₁-C₁₈-alkyl or C₁-C₆-alkoxy, and/or a 5- to 7-membered heterocyclic radical which is bonded via a nitrogen atom and which may comprise further heteroatoms and be aromatic;
C₅-C₈-cycloalkyl whose carbon skeleton may be interrupted by one or more —O—, —S— and/or —NR¹—moieties and/or which may be mono- or polysubstituted by C₁-C₆-alkyl;
aryl or hetaryl which may be mono- or polysubstituted by C₁-C₁₈-alkyl, C₁-C₆-alkoxy, cyano, nitro, halogen, —CONR²R³, —SO₂NR²R³, —COOR², —SO₃R² and/or aryl- or hetarylazo, each of which may be substituted by C₁-C₁₀-alkyl, C₁-C₆-alkoxy or cyano.

3. The process according to claim 1, where the symbols and indices in formula (I) are each defined as follows
n is 0, 1, 2 or 3;
m is an integer from 0 to 2 when n=0;
m is an integer from 0 to 4 when n=1;
m is an integer from 0 to 4 when n=2;
m is an integer from 0 to 6 when n=3;
X is the same or different and is NHR³, phenyloxy, phenylthio, phenylamino, pyrimidyloxy, pyrimidylthio, pyrrolidinyl or piperidinyl, in each case unsubstituted or substituted by one or more radicals from the group of (C₁-C₁₀)-alkyl, (C₁-C₁₀)-alkoxy, cyano, COOR¹ and SO₃R¹;
R¹ is hydrogen or C₁-C₆-alkyl;
R³ is hydrogen, C₁-C₁₈-alkyl which may be substituted by C₁-C₆-alkoxy, halogen, hydroxyl, carboxyl and/or cyano; aryl or hetaryl which may be substituted by the above radicals mentioned for alkyl and by C₁-C₆-alkyl;
and
R is (C₄-C₂₀)-alkyl or phenyl which is unsubstituted or substituted by one or more (C₁-C₁₀)-alkyl groups.

4. The process according to claim 1, where the symbols and indices in formula (I) are each defined as follows:
n is 0, 1, 2 or 3;
m is 0 or 2 when n=0;
m is 0, 2 or 4 when n=1;
m is 0, 2 or 4 when n=2;
m is 0, 2,4 6 when n=3;
X is the same or different and is NH(C₁-C₁₀)-alkyl, phenoxy, phenylamino, pyrrolidinyl, piperidinyl, where the radicals mentioned are unsubstituted or each substituted by from one to three (C₁-C₈)-alkyl groups; and
R is (C₄-C₂₀)-alkyl or phenyl which is substituted by one or more (C₁-C₈)-alkyl groups.

5. The process according to claim 1, where the symbols and indices in formula (I) are each defined as follows:
n is 0, 1, 2 or 3;
m is 0 or 2 when n=0;
m is 0, 2 or 4 when n=1;
m is 0, 2 or 4 when n=2;
m is 0, 2, 4 or 6 when n=3;
X is 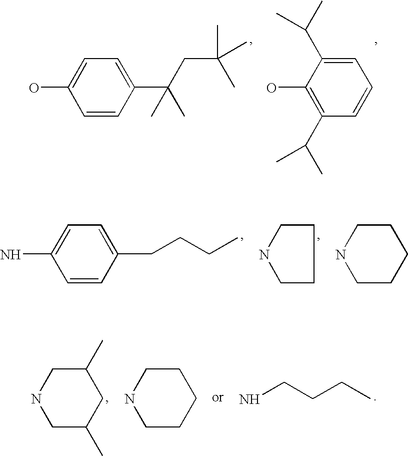
and
R is

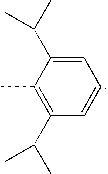

6. A rylene derivative selected from the group consisting of:

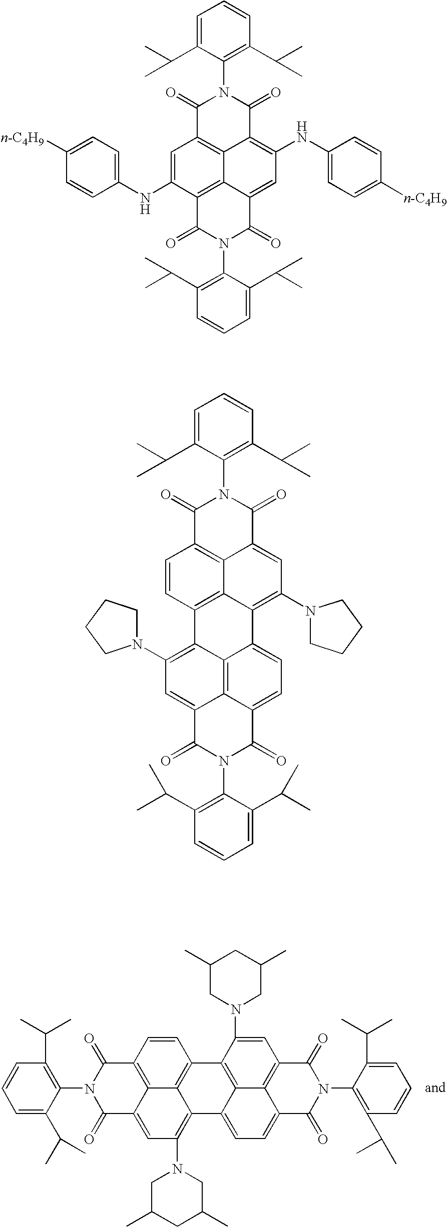

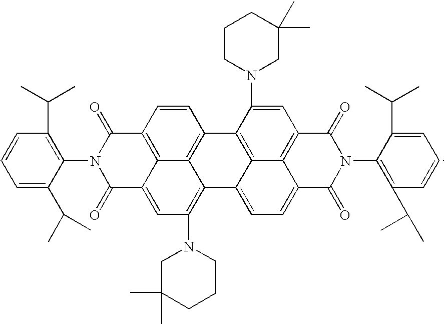

7. A mineral oil comprising at least one compound of formula (I) 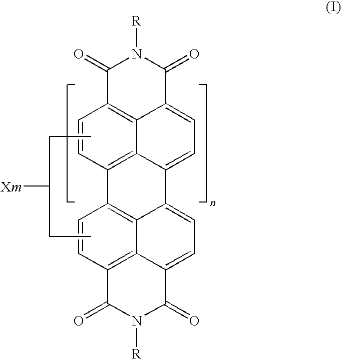
where the symbols and indices are defined as follows
n is an integer from 0 to 3;
m is an integer from 0 to 2 when n=0;
m is an integer from 0 to 4 when n=1;
m is an integer from 0 to 4 when n=2;
m is an integer from 0 to 6 when n=3;
X is the same or different and is
halogen
or
C₁-C₁₀-alkyl, C₃-C₂₀-alken-2-yl or C₃-C₂₀-alkyn-2-yl whose alkyl chain may in each case be interrupted by one or more —O—, —S—, —CO— and/or —SO₂— moieties and which may be mono- or polysubstituted by cyano, C₁-C₆-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic;
or
aryloxy, arylthio, hetaryloxy or hetarylthio, to each of which may be fused further saturated or unsaturated 5- to 7-membered rings whose carbon skeleton may be interrupted by one or more —O—, —S—, —NR¹—, —N═CR¹—, —CO—, —SO— and/or —SO₂— moieties, where the entire ring system may be mono- or polysubstituted by the alkyl radicals (1), cycloalkyl radicals (2), aryl or hetaryl radicals (3) mentioned for R and/or the (i) and/or (iv) radicals mentioned there;
or
a 5- to 9-membered ring which is bonded via a nitrogen atom and whose carbon chain may be interrupted by one or more —O—, —S—, —NR¹—, —CO— and/or —SO₂— moieties, to which may be fused one or two unsaturated or saturated 4- to 8-membered rings whose carbon chain may likewise be interrupted by these moieties and/or —N═, where the entire ring system may be mono- or polysubstituted by: hydroxyl, nitro, —NHR², carboxyl, —COOR², —CONR²R³ or —NR²COR³;
C₁-C₃₀-alkyl whose carbon chain may be interrupted by one or more —O—, —S—, NR¹—, —CO— and/or —SO₂— moieties and which may be mono- or polysubstituted by cyano, hydroxyl, nitro, C₁-C₆-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic;
C₅-C₈-cycloalkyl whose carbon skeleton may be interrupted by one or more —O—,
—S— and/or —NR¹— moieties and/or which may be mono- or polysubstituted by C₁-C₆-alkyl;
aryl or hetaryl, each of which may be mono- or polysubstituted by C1-C18-alkyl, C₁-C₆-alkoxy, cyano, nitro, halogen, —CONR²R³, —NR²COR³, —SO₂NR²R³ and/or aryl- or hetarylazo, each of which may be substituted by C₁-C₁₀-alkyl, C₁-C₆- alkoxy or cyano,
or
C₁-C₁₂-alkoxy, C₁-C₁₂-alkylthio or NR³R⁴;
R¹ is the same or different and is hydrogen or C₁-C₁₈-alkyl,
R², R³, R⁴ are the same or different and are each hydrogen,
C₁-C₁₈-alkyl whose carbon chain may be interrupted by one or more —O—, —S—, —CO—, —SO— and/or —SO₂— moieties and which may be mono- or polysubstituted by C₁-C₁₂-alkoxy, C₁-C₆-alkylthio, hydroxyl, mercapto, halogen, cyano, nitro and/or —COOR¹; aryl or hetaryl, to each of which may be fused further saturated or unsaturated 5- to 7-membered rings whose carbon skeleton may be interrupted by one or more —O—, —S—, —CO— and/or —SO₂— moieties, where the entire ring system may be mono- or polysubstituted by C₁-C₁₂-alkyl and/or the aforementioned radicals mentioned as substituents for alkyl;
and
R is the same or different and is hydrogen,
(1) C₁-C₃₀-alkyl whose carbon chain may be interrupted by one or more —O—,—S—, —NR¹—, —N═CR¹—, —C≡C—, —CR¹═CR¹—, —CO—, —SO— and/or —SO₂— moieties and which may be mono- or polysubstituted by:
(i) C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR² and/or —SO₃R²;
(ii) aryl or hetaryl, to which may be fused further saturated or unsaturated 5- to 7-membered rings whose carbon skeleton may be interrupted by one or more —O—, —S—, —NR¹—, —N═CR¹—, —CR¹═CR¹—, —CO—, —SO— and/or —SO₂— moieties, where the entire ring system may be mono- or polysubstituted by: C₁-C₁₈-alkyl, C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR², —SO₃R², aryl and/or hetaryl, each of which may be substituted by C₁-C₁₈-alkyl, C₁-C₁₂-alkoxy, hydroxyl, mercapto, halogen, cyano, nitro, —NR²R³, —NR²COR³, —CONR²R³, —SO₂NR²R³, —COOR² and/or —SO₃R²;
(iii) C₃-C₈-cycloalkyl, whose carbon skeleton may be interrupted by one or more —O—, —S—, —NR¹—, —N═CR¹—, —CR¹═CR¹—, —CO—, —SO— and/or —SO₂ moieties and to which may be fused further saturated or unsaturated 5- to 7-membered rings whose carbon skeleton may be interrupted by one or more —O—, —S—, —NR¹—, —N═CR¹—, —CR¹═CR¹—, —CO—, —SO— and/or —SO₂— moieties, where the entire ring system may be mono- or polysubstituted by: C₁-C₁₈-alkyl, C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, CONR²R³, —SO₂NR²R³, —COOR² and/or —SO₃R²;
(iv) a —U-aryl radical which may be mono- or polysubstituted by the above radicals mentioned as substituents for the aryl radicals (ii), where U is an —O—, —S—, —NR¹—, —CO—, —SO— or —SO₂— moiety;
(2) C₃-C₈-cycloalkyl to which may be fused further saturated or unsaturated 5- to 7-membered rings whose carbon skeleton may be interrupted by one or more —O—, —S—, —NR¹—, —N═CR¹—, —CR¹═CR¹—, —CO—, —SO— and/or —SO₂— moieties, where the entire ring system may be mono- or polysubstituted by: the (i), (ii), (iii), (iv) radicals and/or
(v) C₁-C₃₀-alkyl whose carbon chain may be interrupted by one or more —O—, —S—, —NR¹—, —N═CR¹—, —C≡C—, —CR¹═CR¹—, —CO—, —SO— and/or —SO₂— moieties and which may be mono- or polysubstituted by: C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR², —SO₃R₂, aryl and/or saturated or unsaturated C₄-C₇-cycloalkyl whose carbon skeleton may be interrupted by one or more —O—, —S—, —NR¹—, —N═CR¹—, —CR¹═CR¹—, —CO—, —SO— and/or —SO₂— moieties, where the aryl and cycloalkyl radicals may each be mono- or polysubstituted by C₁-C₁₈-alkyl and/or the above radicals mentioned as substituents for alkyl;
(3) aryl or hetaryl, to which may be fused further saturated or unsaturated 5- to 7-membered rings whose carbon skeleton may be interrupted by one or more —O—, —S—, —N═CR¹—, —C≡C—, —CR¹═CR¹—, —CO—, —SO— and/or —SO₂— moieties, where the entire ring system may be substituted by the (i), (ii), (iii), (iv), (v) radicals and/or aryl- and/or hetarylazo, each of which may be substituted by C₁-C₁₀-alkyl, C₁-C₆-alkoxy and/or cyano
as a marker.

8. The mineral oil according to claim 7, wherein the mineral oil is a gasoline, diesel or heating oil.

9. (canceled)

10. A process for identifying mineral oil which comprises at least one compound of formula (I) 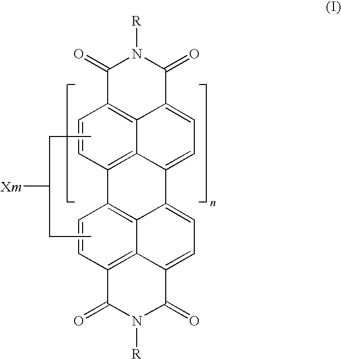
where the symbols and indices are defined as follows
n is an integer from 0 to 3;
m is an integer from 0 to 2 when n=0;
m is an integer from 0 to 4 when n=1;
m is an integer from 0 to 4 when n=2;
m is an integer from 0 to 6 when n=3;
X is the same or different and is
halogen
or
C₁-C₁₀-alkyl, C₃-C₁₀-alken-2-yl or C₃-C₂₀-alkyn-2-yl whose alkyl chain may in each case be interrupted by one or more —O—, —S—, —CO— and/or —SO₂— moieties and which may be mono- or polysubstituted by cyano C₁-C₆-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic;
or
aryloxy, arylthio, hetaryloxy or hetarylthio, to each of which may be fused further saturated or unsaturated 5- to 7-membered rings whose carbon skeleton may be interrupted by one or more —O—, —S—, —NR¹—, —N═CR¹—, —CO—, —SO— and/or —SO₂— moieties, where the entire ring system may be mono- or polysubstituted by the alkyl radicals (1), cycloalkyl radicals (2), aryl or hetaryl radicals (3) mentioned for R and/or the (i) and/or (iv) radicals mentioned there;
or
a 5- to 9-membered ring which is bonded via a nitrogen atom and whose carbon chain may be interrupted by one or more —O—, —S—, —NR¹—, —CO— and/or —SO₂— moieties, to which may be fused one or two unsaturated or saturated 4- to 8-membered rings whose carbon chain may likewise be interrupted by these moieties and/or —N═, where the entire ring system may be mono- or polysubstituted by: hydroxyl, nitro, —NHR², carboxyl, —COOR², —CONR²R³ or —NR²COR³;
C₁-C₃₀-alkyl whose carbon chain may be interrupted by one or more —O—, —S—, NR¹—, —CO— and/or —SO₂— moieties and which may be mono- or polysubstituted by cyano, hydroxyl, nitro, C₁-C₆-alkoxy, —COOR², —CONR²R³, aryl which may be substituted by C₁-C₁₈-alkyl or C₁-C₆-alkoxy, and/or a 5- to 7-membered heterocyclic radical which is bonded via a nitrogen atom and may comprise further heteroatoms and be aromatic;
C₅-C₈-cycloalkyl whose carbon skeleton may be interrupted by one or more —O—,
—S— and/or —NR¹— moieties and/or which may be mono- or polysubstituted by C₁-C₆-alkyl;
aryl or hetaryl, each of which may be mono- or polysubstituted by C1-C18-alkyl, C₁-C₆-alkoxy, cyano, nitro, halogen, —CONR²R³, —NR²COR³, —SO₂NR²R³ and/or aryl- or hetarylazo, each of which may be substituted by C₁-C₁₀-alkyl, C₁-C₆-alkoxy or cyano,
or
C₁-C₁₂-alkoxy, C₁-C₁₂-alkylthio or NR³R⁴;
R¹ is the same or different and is hydrogen or C₁-C₁₈-alkyl,
R², R³, R⁴ are the same or different and are each hydrogen,
C₁-C₁₈-alkyl whose carbon chain may be interrupted by one or more —O—, —S—, —CO—, —SO— and/or —SO₂— moieties and which may be mono- or polysubstituted by C₁-C₁₂-alkoxy, C₁-C₆-alkylthio, hydroxyl, mercapto, halogen, cyano, nitro and/or —COOR¹; aryl or hetaryl, to each of which may be fused further saturated or unsaturated 5- to 7-membered rings whose carbon skeleton may be interrupted by one or more —O—, —S—, —CO— and/or —SO₂— moieties, where the entire ring system may be mono- or polysubstituted by C₁-C₁₂-alkyl and/or the aforementioned radicals mentioned as substituents for alkyl;
and
R is the same or different and is hydrogen,
(1) C₁-C₃₀-alkyl whose carbon chain may be interrupted by one or more —O—, —S—, —NR¹—, —N═CR¹—, —C≡C—, —CR¹═CR¹—, —CO—, —SO— and/or —SO₂— moieties and which may be mono- or polysubstituted by:
(i) C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR² and/or —SO₃R²;
(ii) aryl or hetaryl, to which may be fused further saturated or unsaturated 5- to 7-membered rings whose carbon skeleton may be interrupted by one or more —O—, —S—, —NR¹—, —N═CR¹—, —CR¹═CR¹—, —CO—, —SO— and/or —SO₂— moieties, where the entire ring system may be mono- or polysubstituted by: C₁-C₁₈-alkyl, C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR², —SO₃R², aryl and/or hetaryl, each of which may be substituted by C₁-C₁₈-alkyl, C₁-C₁₂-alkoxy, hydroxyl, mercapto, halogen, cyano, nitro, —NR²R³, —NR²COR³, —CONR²R³, —SO₂NR²R³, —COOR² and/or —SO₃R²;
(iii) C₃-C₈-cycloalkyl, whose carbon skeleton may be interrupted by one or more —O—, —S—, —NR¹—, —N═CR¹—, —CR¹═CR¹—, —CO—, —SO— and/or —SO₂ moieties and to which may be fused further saturated or unsaturated 5- to 7-membered rings whose carbon skeleton may be interrupted by one or more —O—, —S—, —NR¹—, —N═CR¹—, —CR¹═CR¹—, —CO—, —SO— and/or —SO₂— moieties, where the entire ring system may be mono- or polysubstituted by: C₁-C₈-alkyl, C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, CONR²R³, —SO₂NR²R³, —COOR² and/or —SO₃R²;
(iv) a —U-aryl radical which may be mono- or polysubstituted by the above radicals mentioned as substituents for the aryl radicals (ii), where U is an —O—, —S—, —NR¹—, —CO—, —SO— or —SO₂— moiety;
(2) C₃-C₈-cycloalkyl to which may be fused further saturated or unsaturated 5- to 7-membered rings whose carbon skeleton may be interrupted by one or more —O—, —S—, —NR¹—, —N═CR¹—, —CR¹═CR¹—, —CO—, —SO— and/or —SO₂— moieties, where the entire ring system may be mono- or polysubstituted by: the (i), (ii), (iii), (iv) radicals and/or
(v) C₁-C₃₀-alkyl, whose carbon chain may be interrupted by one or more —O—, —S—, —NR¹—, —N═CR¹—, —C≡C—, —CR¹═CR¹—, —CO—, —SO— and/or —SO₂— moieties and which may be mono- or polysubstituted by: C₁-C₁₂-alkoxy, C₁-C₆-alkylthio, —C≡CR¹, —CR¹═CR¹₂, hydroxyl, mercapto, halogen, cyano, nitro, —NR²R³, —NR²COR³, —CONR²R³, —SO₂NR²R³, —COOR², —SO₃R₂, aryl and/or saturated or unsaturated C₄-C₇-cycloalkyl whose carbon skeleton may be interrupted by one or more —O—, —S—, —NR¹—, —N═CR¹—, —CR¹═CR¹—, —CO—, —SO— and/or —SO₂— moieties, where the aryl and cycloalkyl radicals may each be mono- or polysubstituted by C₁-C₁₈-alkyl and/or the above radicals mentioned as substituents for alkyl;
(3) aryl or hetaryl, to which may be fused further saturated or unsaturated 5- to 7-membered rings whose carbon skeleton may be interrupted by one or more —O—, —S—, —N═CR¹—, —C≡C—, —CR¹═CR¹—, —CO—, —SO— and/or —SO₂— moieties, where the entire ring system may be substituted by the (i), (ii), (iii), (iv), (v) radicals and/or aryl- and/or hetarylazo, each of which may be substituted by C₁-C₁₀-alkyl, C₁-C₆-alkoxy and/or cyano
in an amount which is sufficient to excite detectable fluorescence with radiation of a suitable wavelength, by
a) irradiating the mineral oil with electromagnetic radiation of a wavelength of from 600 to 1000 nm and
b) detecting the excited fluorescence radiation with an apparatus for detecting radiation in the range from 600 to 1200 nm.
